(19) United States
(12) Patent Application Publication
(10) Pub. No.: US 2025/0356972 A1
(43) Pub. Date: Nov. 20, 2025

(54) METHODS AND SYSTEMS THAT PROVIDE PERSONALIZED MEDICAL TREATMENTS BY DEFORMING GENERIC EFFICACY-ESTIMATION FUNCTIONS

(71) Applicant: Craig Alan Friedman, new York, NY (US)

(72) Inventor: Craig Alan Friedman, new York, NY (US)

(21) Appl. No.: 18/831,519

(22) Filed: Mar. 12, 2025

Related U.S. Application Data
(60) Provisional application No. 63/564,119, filed on Mar. 12, 2024.

(51) Int. Cl. G16H 20/00; G16H 70/20

(52) U.S. Cl. CPC G16H 20/00; G16H 70/20

(57) ABSTRACT

The current document is directed to methods and systems that generate personalized treatment and therapy plans for patients. Currently disclosed implementations of these methods and systems maintain one or more databases that store general patient information as well as information about different types of treatments and therapies, including generic and patient-specific efficacy models that provide estimates of the efficacy of a treatment plan prior to application of the treatment encoded in the treatment plan. Based on the results of a limited number of experiments conducted on a particular patient, on extensive treatment histories for large numbers of patients, and/or on the treatment history for the particular patient, the currently disclosed methods and systems generate a treatment plan by deforming a generic efficacy model and then using the deformed model to identify optimal or near-optimal values for control variables that together represent the treatment plan.

### CROSS-REFERENCE TO RELATED APPLICATION

This application claims the benefit of Provisional Application No. 63/564,119, filed Mar. 12, 2024, which is incorporated in its entirety, by reference.

### TECHNICAL FIELD

The current document is directed to personalized medicine and, in particular, to methods and systems that determine and provide personalized medical treatments and therapies to patients.

### BACKGROUND

There are many different types of treatments and therapies provided to patients suffering from many different types of diseases, pathologies, and disorders. Therapies and treatments may include application of heat and cold, electromagnetic radiation, mechanical forces, and other forces to all or portions of patients' bodies, provision of information and feedback to patients through various means of communication, provision of pharmaceuticals that are ingested, received by injection, inhaled, or delivered to patients by various additional means, surgical interventions, and many other types of therapies. Medical therapies and treatments, including pharmaceuticals, are often thoroughly tested for efficacy and safety before they are allowed to be administered to patients. However, much of this testing is statistical in nature and does not reflect the particular and specific characteristics of individual patients. During the past several decades, it has become increasingly clear that each human being is genetically unique and that medical therapies deemed safe and effective for patients in general may vary considerably in effectiveness and safety among individual patients. These realizations, combined with rapidly evolving technologies for sequencing genomes and acquiring detailed molecular and physiological characterizations of individual patients, have resulted in increasing efforts to personalize medical diagnosis and medical therapies. However, the desire and great effort expended to develop and commercialize personalized medicine are still in the early stages of development and application. In particular, for many types of treatments and therapies, the complexities of evaluating the safety and efficacy of these therapies with respect to individual patients has rendered many of the current approaches to personalized medicine impractical or infeasible. Medical researchers, medical providers, pharmaceutical developers and manufacturers, and developers of therapy-delivering medical systems and methods therefore continue to seek different and effective approaches to providing personalized therapies to patients.

### SUMMARY

The current document is directed to methods and systems that generate personalized treatment and therapy plans for patients. Currently disclosed implementations of these methods and systems maintain one or more databases that store general patient information as well as information about different types of treatments and therapies, including generic and patient-specific efficacy models that provide estimates of the efficacy of a treatment plan prior to application of the treatment encoded in the treatment plan. Based on the results of a limited number of experiments conducted on a particular patient, on extensive treatment histories for large numbers of patients, and/or on the treatment history for the particular patient, the currently disclosed methods and systems generate a treatment plan by deforming a generic efficacy model and then using the deformed model to identify optimal or near-optimal values for control variables that together represent the treatment plan.

### BRIEF DESCRIPTION OF THE DRAWINGS

FIG. 1 illustrates logical components of the currently disclosed personalized-medical-treatment systems.

FIG. 2 illustrates certain fundamental logical entities and functions fundamental to the implementations of the medical-treatment methods and systems disclosed in the current document.

FIG. 3 illustrates examples of the types of data stored in the database or databases maintained by the currently disclosed methods and systems.

FIGS. 4A-D provide a control-flow diagram that illustrates one implementation of the currently disclosed methods that is implemented by the currently disclosed systems.

FIGS. 5A-B illustrate one implementation of the deformation process by which a generic efficacy-estimation function is modified to produce a more accurate patient-specific efficacy-estimation function.

FIG. 6 illustrates, using a 1-dimensional example, the deformation or modification of a generic efficacy-estimation function to produce a patient-specific efficacy-estimation function.

FIGS. 7A-B illustrate the deformation process introduced above with reference to FIG. 6.

FIG. 8 illustrates a technique used, in certain implementations of the currently disclosed methods and systems, to expand the search space of control-variable vectors explored in the constrained optimization/minimization processes discussed above with reference to FIGS. 7A-B.

FIG. 9 illustrates initial steps in batch or sample preparation.

FIG. 10 illustrates additional initial processing steps used to generate signal samples and batches.

FIG. 11 illustrates generation of a batch, for training, or a sample for classification.

FIG. 12 illustrates one implementation of the convolutional neural network that implements the example severity function disclosed in the current document.

FIGS. 13A-B and 14 illustrate details of individual layers of the convolutional neural network that implements the example severity function disclosed in the current document.

FIG. 15 provides a general architectural diagram for various types of computers.

FIG. 16 illustrates an Internet-connected distributed computing system.

FIG. 17 illustrates cloud computing.

FIG. 18 illustrates generalized hardware and software components of a general-purpose computer system, such as a general-purpose computer system having an architecture similar to that shown in FIG. 1.

FIGS. 19A-B illustrate two types of virtual machine and virtual-machine execution environments.

FIG. 20 illustrates fundamental components of a feed-forward neural network.

FIGS. 21A-J illustrate operation of a very small, example neural network.

FIGS. 22A-C show details of the computation of weight adjustments made by neural-network nodes during backpropagation of error vectors into neural networks.

FIGS. 23A-B illustrate neural-network training.

FIGS. 24A-F illustrate a matrix-operation-based batch method for neural-network training.

FIGS. 25A-C illustrate various aspects of recurrent neural networks.

FIGS. 26A-C illustrate a convolutional neural network.

### DETAILED DESCRIPTION

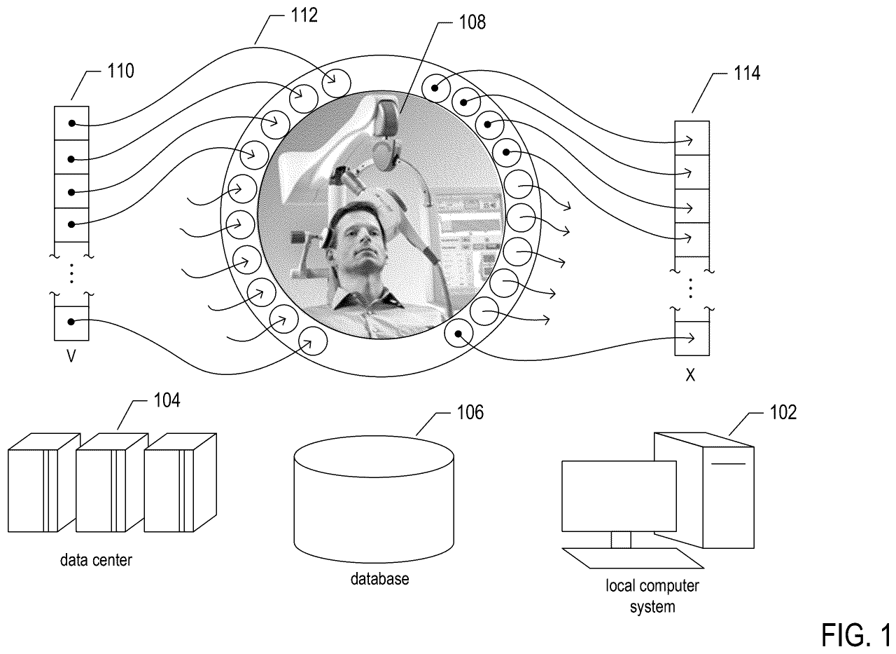

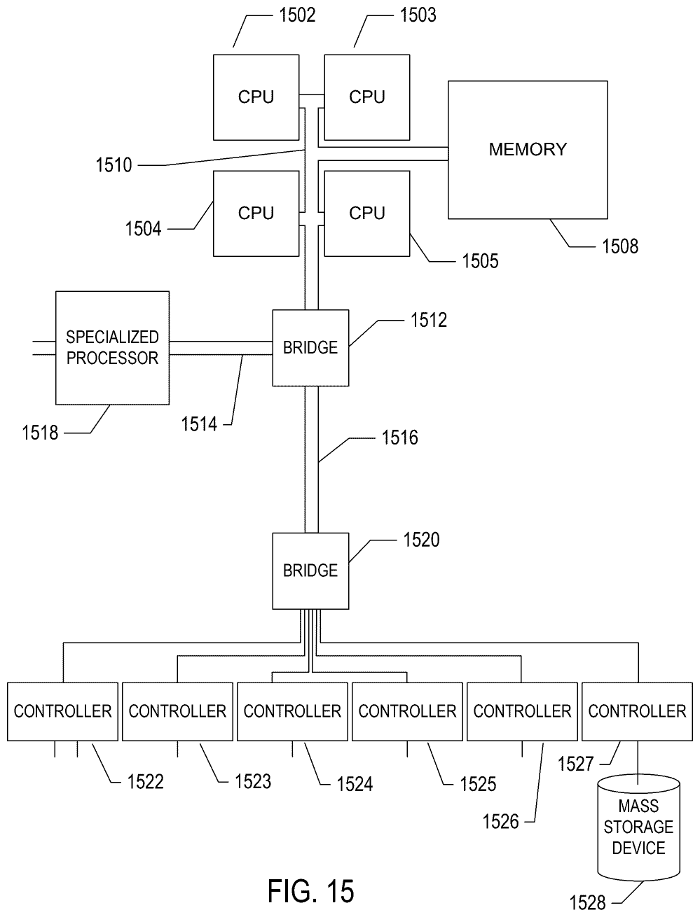

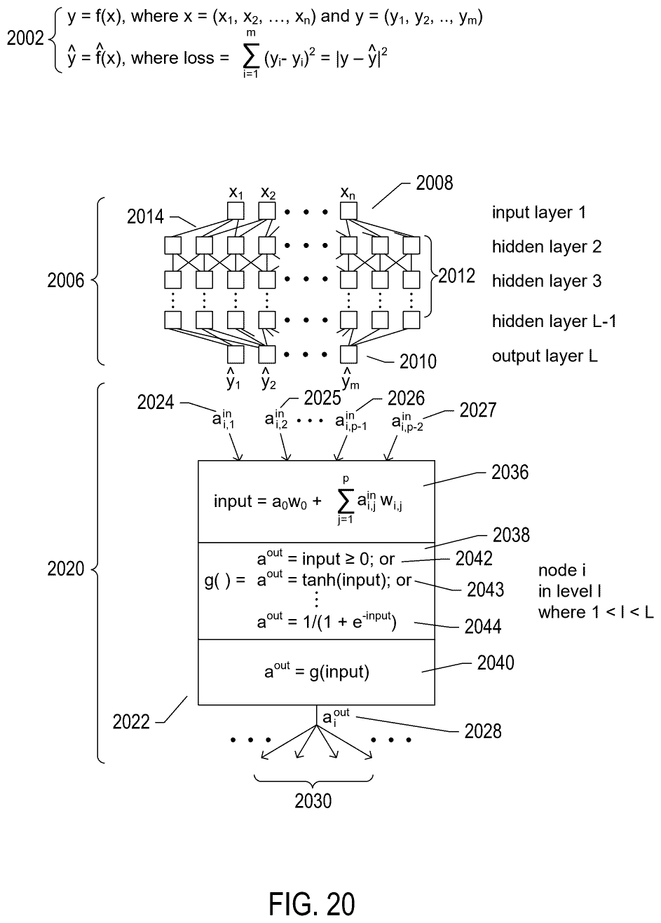

The current document is directed to methods and systems that generate and apply personalized treatment and therapy plans to patients. A first subsection, below, discusses the currently disclosed methods and systems and explains an implementation of the currently disclosed methods and systems with reference to FIGS. 1-14. An overview of computer hardware, complex computational systems, operating systems, and virtualization is provided with reference to FIGS. 15-19B. A third subsection provides an overview of neural networks with reference to FIGS. 20-26C.

### Currently Disclosed Methods and Systems

FIG. 1 illustrates logical components of the currently disclosed personalized-medical-treatment systems. A personalized medical-treatment system may include a local computer system 102, a remote computer system 104, such as a data center or cloud-computing facility, or both a local computer system and a remote computer system. Medical-treatment applications running with one or more of the computer systems control generation of treatment plans using stored data, including patient information, treatment histories, and various models and functions, discussed below. The data is stored in a database 106 or multiple databases accessible to one or both of the local computer system and remote computer system. The system is incorporated in a medical-treatment facility or medical-therapy facility that includes one or more of a wide variety of different types of treatment systems and devices. Each different type of treatment system and device, such as the treatment device 108 shown in FIG. 1, is associated with a set of control variables that are directly input into, or used to generate or derive direct inputs for, the treatment devices and systems. Control variables may include instructions and directions to treatment providers and therapists. A control-variable vector v 110 contains values for controlling or instructing devices, systems and personnel to apply a particular type of treatment to a particular patient, and thus represents a treatment plan or a therapy plan. In FIG. 1, curved arrows, such as curved arrow 112, represent input of control-variable-vector values to devices, systems, and personnel within a treatment facility to effect a treatment or therapy.

The medical-treatment facility also includes various different types of electromechanical monitors, human observers, and patient-response-prompting methods and facilities which produce observations that are encoded into a logical vector of observation data X 114. Observations are used to evaluate a patient prior to treatment and to determine the efficacy of a treatment after it has been applied. The exact types and formats of the control-variable values and observational data may vary widely among different types of treatment devices and treatment facilities. However, for descriptive purposes, the control-variable values and observational data are treated as floating-point values in the following discussion.

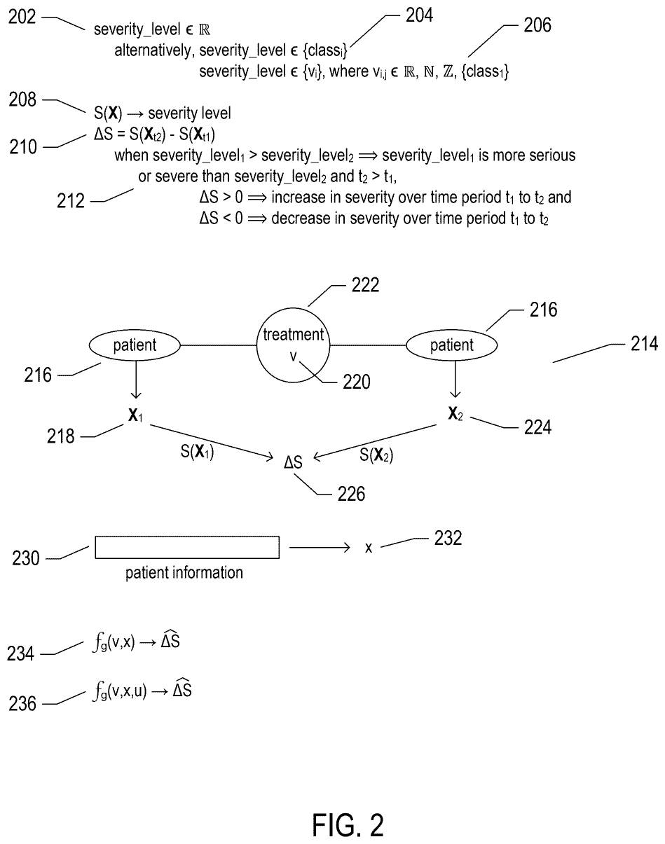

FIG. 2 illustrates certain fundamental logical entities and functions fundamental to the implementations of the medical-treatment methods and systems disclosed in the current document. A severity level 202 represents the severity, seriousness, or undesirability of a medical condition, pathology, or mental state. In the current discussion, severity levels are assumed to be encoded as floating-point numbers, although they may alternatively be encoded as indications of a class within a set of classes 204 or as vectors with multiple components of different types 206. In different implementations, different types of numerical and non-numerical values may be used to represent severity levels. A severity function S(X) 208 receives an observation-data vector and returns a severity-level value corresponding to the observation data. A change in severity level ΔS 210 is computed as the difference between a severity level computed from observation data acquired at a time t2 and a severity level computed from observation data at a time t1, where t2 is later than t1 (t2>t1). Assuming that increasing positive values of severity levels indicate increasing severity, seriousness, or undesirability, a positive ΔS indicates a deterioration in a patient's condition and a negative ΔS indicates an improvement 212 in a patient's condition.

A fundamental cycle in the provision of medical treatment and medical therapies is shown in diagram 214 in the middle of FIG. 2. A patient 216 is initially observed and/or monitored and the observations are encoded in a first observation-data vector 218. A treatment or therapy encoded in a control-variable vector v 220 is then applied to the patient 222. Following the treatment or therapy, the patient 216 is again observed and/or monitored and the observations are encoded in a second observation-data vector 224. Finally, a ΔS value 226, or treatment-efficacy estimate, is determined for the treatment by subtracting the severity level computed from the first observation-data vector from the severity level computed from the second observation-data vector. Thus, a ΔS value is a measure of the efficacy of a treatment applied to a patient and may be variously referred to as an “efficacy estimate,” an “observed efficacy,” or as a “treatment result.”

During the course of evaluating and providing treatments and therapies to patients, patient information that describes and characterizes the patient is collected and stored in one or more databases. In general, patient information may be numeric, textual, or encoded in other types of information-containing forms. Patient information 230 can be processed to generate a vector x 232 containing encoded patient-specific and treatment-specific information that is used in generating treatment plans, as discussed below. Such vectors are referred to as (“pti vectors”) in the following discussion. The information encoded in a pti vector is that information which is needed to generate and evaluate treatment plans. Finally, two different types of efficacy-estimation models or functions are used in generating treatment plans. A first type of efficacy-estimation function 234 receives a control-variable vector representing a treatment plan and a pti vector representing a specific patient and returns an estimate of the efficacy that would be obtained by treating the patient according to the treatment plan. This type of efficacy-estimation function is referred to as a “generic efficacy-estimation function” (“f_{g}”) because the function is generated from historic patient-treatment data collected from many different patients and is not specific to a particular patient, but generic efficacy-estimation functions do provide reasonable estimates for any particular patient since they incorporate knowledge acquired over extensive time periods and across many different patients. By contrast, a second type of efficacy-estimation function 236 also receives a control-variable vector representing a treatment plan and a pti vector representing a particular patient and returns an estimate of the efficacy that would be obtained by treating the particular patient according to the treatment plan, but the second type of efficacy-estimation function has an implicit third argument u representing information specific to the particular patient described by the second observation-data vector argument. The implicit third argument is not input as an argument because it is generally not known. The third argument is simply an indication that the second type of efficacy-estimation function incorporates additional patient-specific information that may not be directly incorporated into a generic efficacy-estimation function. This second type of efficacy-estimation function is referred to as a “patient-specific efficacy-estimation function” (“fₚ”) because, although the function is generated from historic patient-treatment data collected from many different patients, a patient-specific efficacy-estimation function incorporates additional patient-specific information represented by the implicit third argument u. This additional patient-specific information may include experimentally derived information, as further discussed below, but it is generally not known and not explicitly represented and the implicit third argument u is not input as an argument when the patient-specific efficacy-estimation function is called or invoked.

In the current discussion, the terms “model” and “function” used in the phrases “generic efficacy-estimation function,” “generic efficacy-estimation model,” “patient-specific efficacy-estimation function,” and “patient-specific efficacy-estimation model” are interchangeable, having the same meaning. The term “function” is used more frequently. These models/functions can be implemented in many different ways, including by neural networks, transformers, large-language models, rule-based systems, decision-tree-based systems, and other such technologies and combinations of technologies, although, in general, they need to be trainable from treatment-history data.

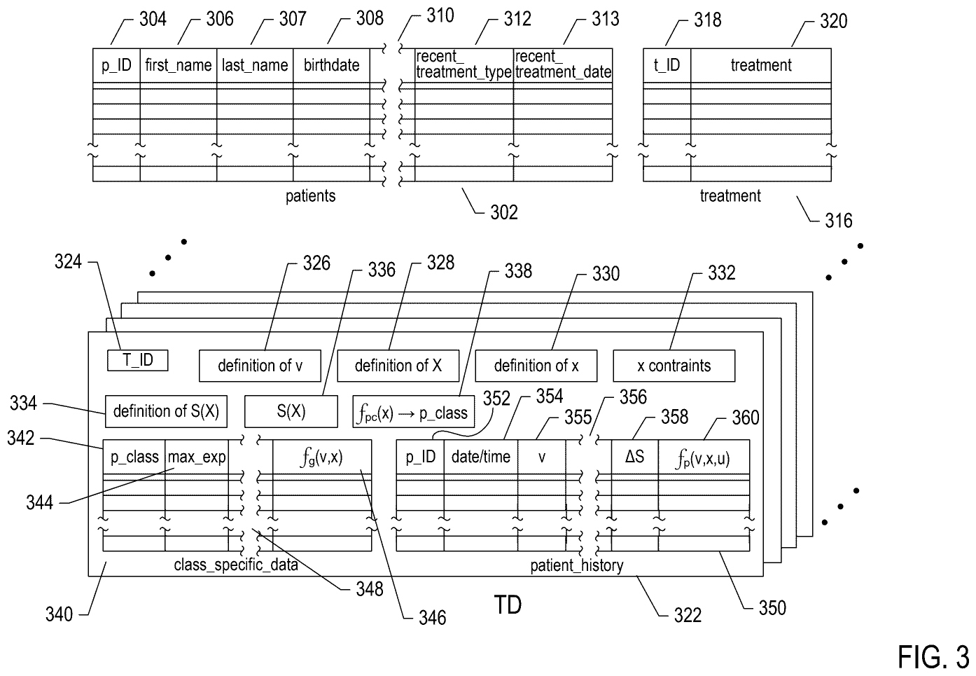

FIG. 3 illustrates examples of the types of data stored in the database (106 in FIG. 1) or databases maintained by the currently disclosed methods and systems. Different implementations may use any of various different types of databases and other data-storage technologies. For simplicity, the data is shown as relational database tables and discrete data entities in FIG. 3. Patient data is stored in a table patients 302. The patient data stored for a given patient, represented as a row in the table patients, may include a unique patient identifier 304, first and last names 306-307, a birthdate 308, many additional types of patient-specific information, such as an address, insurance information, and a health history, represented in FIG. 3 by broken column 310, and an indication of the most recent type of treatment received by the patient and the date of that treatment 312-313. A table treatment 316 represents different types of treatment, each treatment type associated with an identifier 318 and a textual description 320. The database may include many additional types of data not shown in FIG. 3 since such data is not directly relevant to the current discussion.

In the. currently disclosed implementation, each specific type of treatment carried out using a particular type of treatment device or facility is represented in the database by a collection of data referred to as a “treatment descriptor” (“TD”), one example of which 322 is illustrated in FIG. 3. The TD includes: (1) a treatment identifier 324; (2) a definition of the control variables included in a control-variable vector for a treatment plan for the treatment type 326; (3) a definition of the observation-data encodings included in an observation-data vector for the treatment type 328; (4) a definition of the pti vector used to encode patient-specific and treatment-specific information for specific patients 330; (5) a definition of any treatment-type constraints that may be associated with patient-specific and treatment-specific information 332; (6) a definition of the severity function for the treatment type 334; (7) the severity function for the treatment type 336; and (8) a patient-class function 338 that receives a pti vector representing a particular patient and returns the identifier for a patient class with which that patient is associated. In addition, the TD includes a table class_specific_data 340, each row of which represents a patient class with respect to the treatment type, each row including a patient-class identifier 342, a maximum number of experiments for the patient class 344, a generic efficacy-estimation function for the patient class 346, and other class-specific data represented by broken column 348. The TD also includes a table patient_history 350, each row of which represents patient-specific information relevant to the treatment type, each row including a patient identifier 352, the date and time of a treatment 354, the control-variable vector representing the treatment plan for the treatment 355, additional data represented by broken column 356, the treatment result indicated as a ΔS value 358, and a patient-specific efficacy-estimation function that was used to determine the treatment plan for the treatment 360. The TD for any particular implementation may include additional data values not shown in FIG. 3 and may omit certain of the data values shown in FIG. 3. As one example, many implementations do not make use of patient-class-specific generic efficacy-estimation functions but instead use a single generic efficacy-estimation function for all patients. In those implementations, the single generic efficacy-estimation function provides sufficient accuracy across all patients. In fact, in certain cases, a single generic efficacy-estimation function may be used for multiple different related treatment types. Many implementations may not use any patient-class-specific data. Note also that there may be multiple TDs for any given treatment type, since the data stored in a TD is specific not only for the treatment type but also for a specific, or specific class of, treatment devices and/or facilities. Moreover, multiple different treatment devices or systems may be represented by a single TD for a particular treatment type when they share similar control-variable-vector and observation-data-vector definitions.

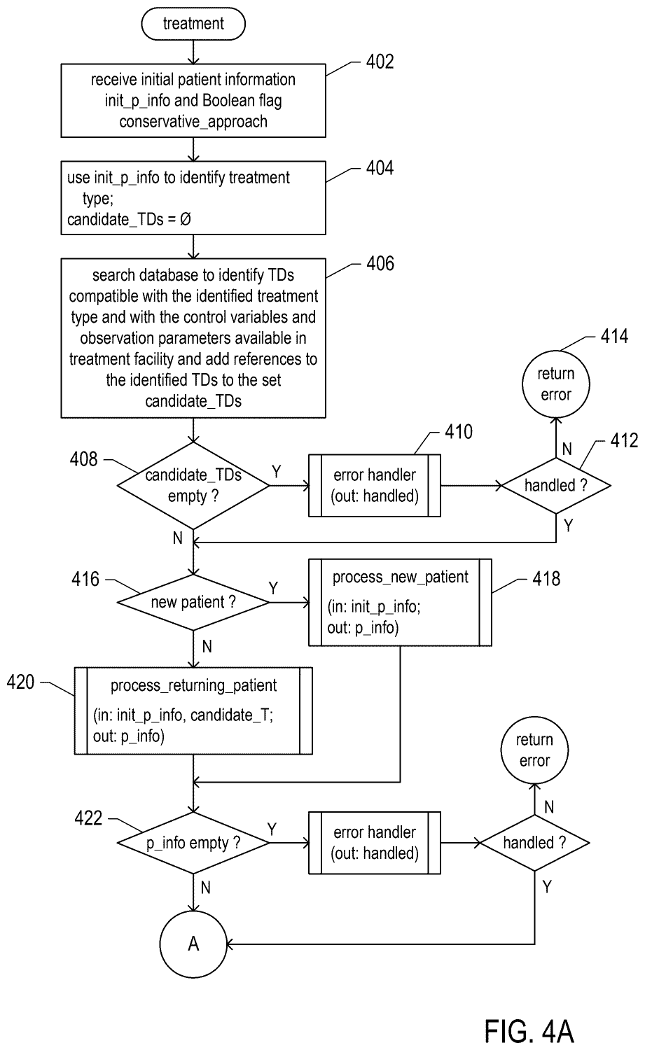

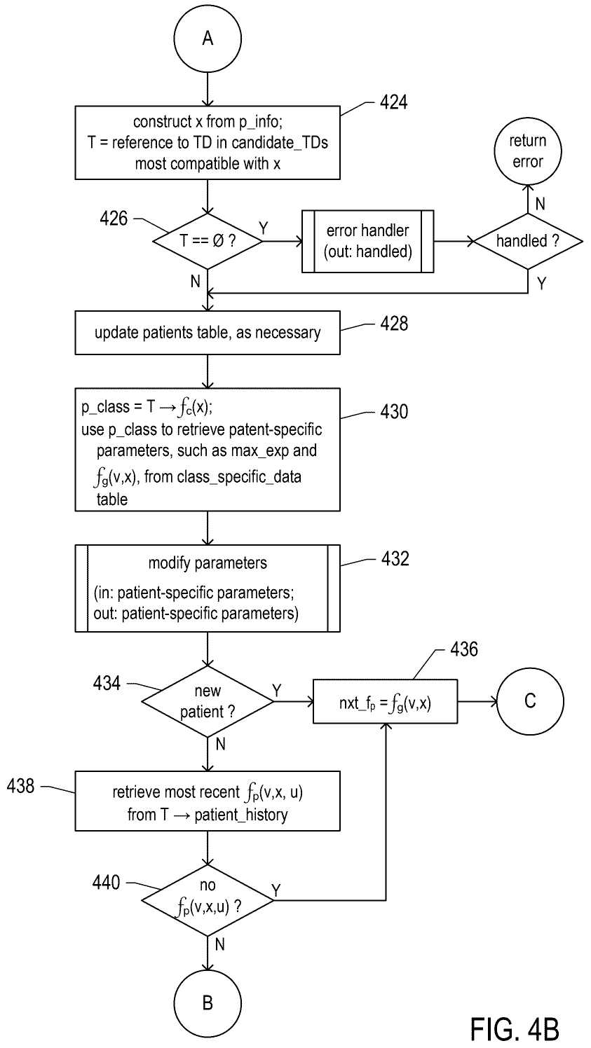

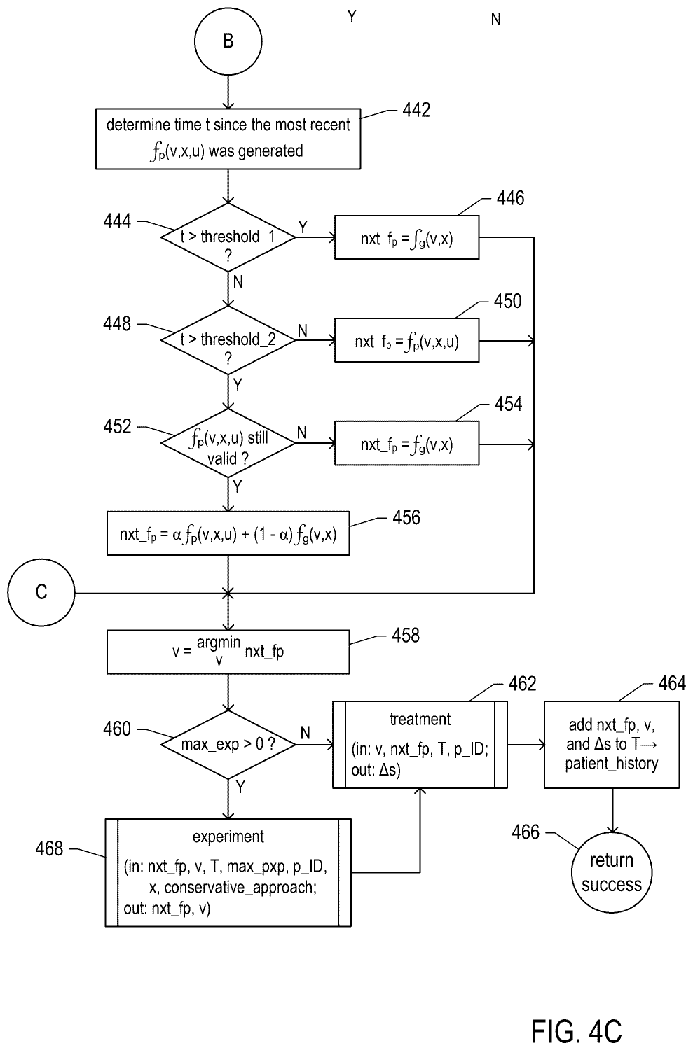

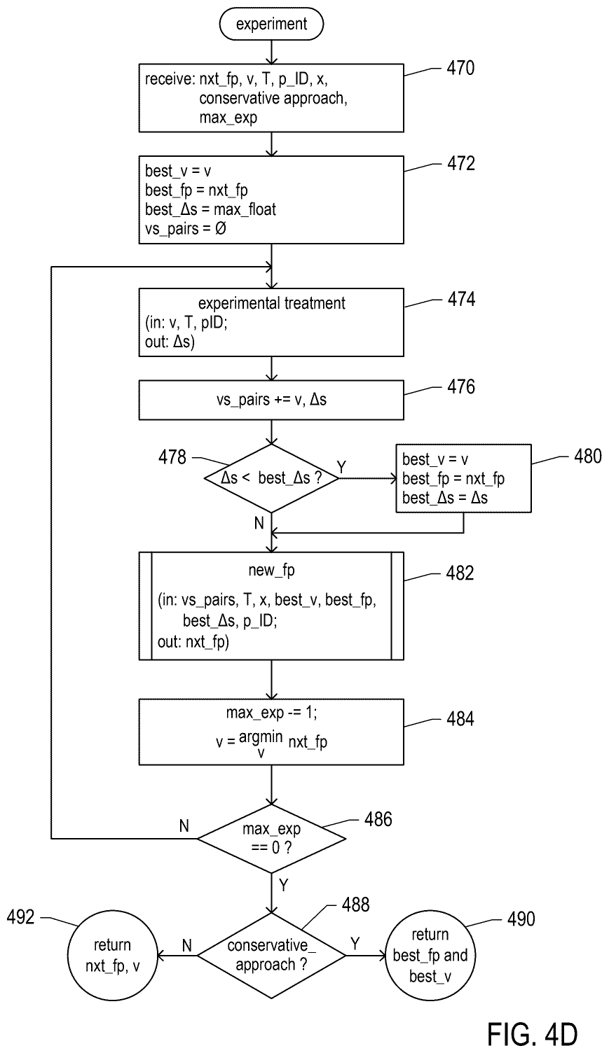

FIGS. 4A-D provide a control-flow diagram that illustrates one implementation of the currently disclosed methods that is implemented by the currently disclosed systems. FIG. 4A shows an initial portion of the control-flow diagram for a routine “treatment.” In step 402, the routine “treatment” receives initial patient information init_p_info and a Boolean flag conservative_approach. The initial patient information is information either provided by an automated system, such as an automated treatment-scheduling system, or by the patient in cooperation with treatment-facility personnel. The specific data content of the init_p_info may vary from implementation to implementation, from time to time, and from patient to patient. This information may include identifying information for the patient as well as information indicating the type of treatment desired or needed by the patient. The Boolean flag conservative_approach indicates whether treatment plans should be generated using an aggressive approach or a more conservative approach, as further discussed below. In step 404, the routine “treatment” uses the received init_p_info information to determine a treatment type and initializes a set of candidate_TDs to the empty set. In step 406, the routine “treatment” searches the database to identify TDs compatible with the identified treatment type and with the types of control variables and observation data associated with the treatment device and/or treatment facility. This search considers the definitions 326 and 328 included in the TDs, discussed above with reference to FIG. 3. References to compatible TDs are stored in the set candidate_TDs. If no compatible TD is found, as determined in step 408, an error handler is called in step 410. If the failure to identify a compatible TD is not handled by the error handler, as determined in step 412, the routine “treatment” returns an error in step 414. This same type of error handling is shown in additional portions of the control-flow diagram and will not be repeatedly discussed. Furthermore, much additional error handling may be incorporated into any particular implementation.

When the patient is a new patient, as determined from the init_p_info information in step 416, a routine process_new_patient is called, in step 418, to generate a full data description for the patient p_info. This routine represents a process of extracting further information from the patient via written forms, verbal inquiries, and other means. Otherwise, a routine process_returning_patient is called, in step 420, in order to obtain sufficient information to retrieve patient information from the table patients in the database, supplemented and updated, as necessary, in order to generate a full data description for the patient p_info. When an adequate p_info data description has not been generated via one of the two routines process_new_patient and process_returning_patient, as determined in step 422, an error is raised and handled. Control flows to the top of FIG. 4B.

Turning to FIG. 4B, in step 424, the routine “treatment” constructs a pti vector from the data description p_info, as mentioned with respect to items 230 and 232 in FIG. 2, and then searches the TD references in the set candidate_TDs to identify the TD most compatible with the contents of the a pti vector, with the variable T set to reference the most compatible TD. If no compatible TD is found, as determined in step 426, an error is raised and handled. In step 428, the table patients is updated, as necessary, using the data description p_info. In step 430, a patient class p_class is determined for the patient using the patient-class function contained in the identified TD referenced by T, and p_class is used to retrieve patient-specific parameters, such as a maximum number of experiments max_exp and a class-specific generic efficacy-estimation function f_{g} from the table class_specific_data in the TD referenced by T. In step 432, a routine modified parameters is called in order to further modify any of the already identified parameters in accordance with any additional information or observations related to the patient. As one example, treatment-facility personnel may determine that the patient appears to be unwell or in distress and therefore unlikely to benefit from treatment or therapy experimentation, discussed below, as a result of which the parameter max_exp may be set to 0. When the patient is a new patient, as determined from the data description p_info in step 434, a variable nxt_fₚ is set, in step 436, to reference the generic efficacy-estimation function retrieved in step 430, above, since there is no patient-specific efficacy-estimation function for the patient. Control then flows to point C in the control-flow-diagram portion shown in FIG. 4C. Otherwise, in step 438, the routine “treatment” retrieves the most recent patient-specific efficacy-estimation function for the patient from the table patient_history in the TD referenced by variable T. When no patient-specific efficacy-estimation function for the patient has been retrieved, as determined in step 440, control flows to step 436, discussed above. Otherwise, control flows to point B in the control-flow-diagram portion shown in FIG. 4C.

Turning to FIG. 4C, the routine “treatment,” in step 442, determines the length of time t since the most recent patient-specific efficacy-estimation function fₚ retrieved in step 438 was generated using additional information obtained from the table patient_history in the TD referenced by variable T. When t is greater than a first threshold value, as determined in step 444, indicating that sufficient time has elapsed since the generation of the patient-specific efficacy-estimation function fₚ to consider fₚ to be no longer valid, the variable nxt_fₚ is set, in step 446, to reference the generic efficacy-estimation function f_{g} retrieved in step 430. Otherwise, when t is less than a second threshold value, as determined in step 448, indicating that the patient-specific efficacy-estimation function fₚ is likely still optimal or near optimal, the variable nxt_fₚ is set, in step 450, to reference the patient-specific efficacy-estimation function fₚ retrieved in step 438. When the patient-specific efficacy-estimation function fₚ retrieved in step 438 is determined, in step 452, to be invalid for other reasons, such as an invalidating change in the current pti vector generated in step 424 with respect to the pti vector used in the treatment session associated with the patient-specific efficacy-estimation function fₚ retrieved in step 438, then the variable nxt_fₚ is set, in step 454, to reference the generic efficacy-estimation function f_{g} retrieved in step 430. Otherwise, in step 456, the variable nxt_fₚ is set to a new efficacy-estimation function fₚ generated from the patient-specific efficacy-estimation function fₚ retrieved in step 438 and the generic efficacy-estimation function f_{g} retrieved in step 430. This may involve a combination equivalent to a linear weighted combination of the two efficacy-estimation functions. In step 458, a control-variable vector v is generated by optimizing v to produce a lowest ΔS value when input as an argument to the efficacy-estimation function referenced by local variable nxt_fₚ. Any of many different optimization methods, such as gradient-descent methods, can be used to determine an optimal or near-optimal control-variable vector v. An optimization method is essentially a search for the control-variable vector that, when input to the efficacy-estimation function referenced by local variable nxt_fₚ, produces a ΔS result that is less than or equal to the ΔS result produced by all other control-variable vectors. In many practical contexts, where the search space is too large, acceptable optimizations may be local minima rather than a global minimum. When the parameter max_exp is equal to 0, as determined in step 460, a routine “treatment” is called, in step 462, to apply a treatment of the determined treatment type according to the treatment plan represented by the control-variable vector v to the patient using a treatment device and/or treatment facility compatible with the TD referenced by T. The routine “treatment” returns a ΔS result which is entered, along with the control-variable vector v and the efficacy-estimation function referenced by variable nxt_fₚ, in step 464 into the table patient_history in the TD referenced by local variable T. The routine “treatment” then returns a success indication in step. 466. When the parameter max_exp is not equal to 0, as determined in step 460, a routine “experiment” is called, in step 468, to conduct one or more treatment experiments on the patient in order to optimize the treatment plan prior to carrying out the treatment in step 462. In many cases, experimental treatments may differ significantly from treatments represented by the routine “treatment.” As one example, experimental treatments may be applied for a much shorter length of time.

A control-flow diagram for the routine “experiment,” called in step 468 of FIG. 4C, is shown in FIG. 4D. In step 470, the routine “experiment” receives the arguments passed in the call to the routine in step 468 in FIG. 4C. In step 472, local variable best_v is set to the control-variable vector v received in step 470, local variable best_fₚ is set to reference the efficacy-estimation function referenced by nxt_fₚ received in step 470, local variable best_ΔS is set to a large positive value, and the set variable vs_pairs is initialized to the empty set. In step 474, a routine “experimental treatment” is called to apply an experimental version of the treatment corresponding to the treatment plan represented by control-variable vector v to the patient. Following experimental treatment, in step 476, the control-variable vector v and the ΔS result returned by the routine “experimental treatment” are added to the set vs pairs. When the ΔS result returned by the routine “experimental treatment” is less than the values stored in local variable best_ΔS, as determined step 478, then, in step 480, best_v is set to the control-variable vector v, best_fₚ is set to reference the efficacy-estimation function referenced by best_fₚ, and best_ΔS is set to the ΔS result returned by the routine “experimental treatment.” In step 482, a routine “new_fp” is called to generate a new, modified patient-specific efficacy-estimation function based on the accumulated experimental results by a deformation process, as discussed further below. Then, in step 484, the parameter max_exp is decremented and a new control-variable vector v is obtained by an optimization process using the new, modified patient-specific efficacy-estimation function generated in step 482. When the parameter max_exp is greater than 0, as determined in step 486, control returns to step 474 for carrying out an additional experimental treatment using the new treatment plan represented by the new control-variable vector v generated in step 484. Otherwise, in step 488, the routine “experiment” determines whether or not the conservative approach for treatment should be taken based on the value of the Boolean flag conservative_approach. When the conservative approach is to be taken, the routine “experiment” returns, in step 490, the reference stored in local variable and best_fₚ and the control-variable vector referenced by local variable best_v. Otherwise, in step 492, the routine “experiment” returns the reference stored in local variable and next_fₚ and the control-variable vector referenced by local variable best_v.

The routine “treatment,” discussed above and shown in FIGS. 4A-D, represents a typical treatment session. Patients may be treated repeatedly, over the course of many treatment sessions. The exact details of any particular treatment may vary from the typical treatment session depicted in FIGS. 4A-D, due to differences in types of treatment, differences between human patients and human treatment-facility personnel, advances in treatment devices and systems, and for other reasons.

In summary, the currently disclosed methods and systems are designed to provide personalized medical treatments and therapies to a new patient by initially using a generic efficacy-estimation function developed from stored treatment-history data for many patients to generate an initial treatment plan for the new patient. This allows the currently disclosed methods and systems to take advantage of a large amount of accumulated patient-treatment data, which likely includes patient-treatment data for patients similar to the new patient, to generate an initial treatment plan for the new patient that is likely at least reasonably effective and often quite effective. Similarly, the currently disclosed methods and systems are able to use previously generated patient-specific efficacy-estimation functions for returning patients to generate very effective, personalized treatment plans for returning patients. When possible, limited experimentation is used to generate experimental results that are used to deform the generic efficacy-estimation function initially used to generate an initial treatment plan for a patient in order to produce increasingly accurate and updated patient-specific efficacy-estimation functions for both new and returning patients. The increasingly accurate and updated patient-specific efficacy-estimation functions can then be used to generate increasingly effective treatment plans for both new and returning patients. This approach is taken because, unlike in traditional optimization problems, it is not possible, in medical-therapy and medical-treatment contexts, to carry out a sufficient number of experiments for a typical gradient-descent optimization or for other commonly used types of optimizations that depend on stepwise exploration of a generally high-dimensional manifold, to identify near-optimal and optimal patient-specific efficacy-estimation functions. The number of experiments that can be reasonably conducted on a patient varies from treatment type to treatment type, but is usually constrained by time, cost, inconvenience to patients, and often by accumulation of risk associated with each experimental procedure. An approach based on deforming a current generic or patient-specific efficacy-estimation function to generate an improved patient-specific efficacy-estimation function based on a small number of experimental results allows the currently disclosed methods and systems to improve the efficacy of treatment plans for particular patients without violating the significant medical-context constraints on the number of experimental applications of therapies and treatments that can be conducted in order to improve the efficacy of treatment plans for particular patients.

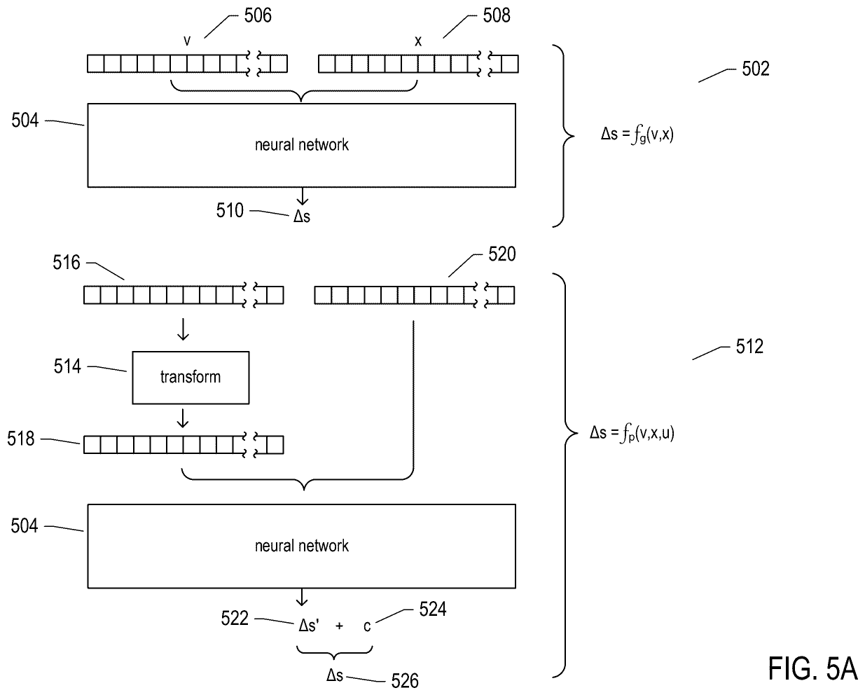

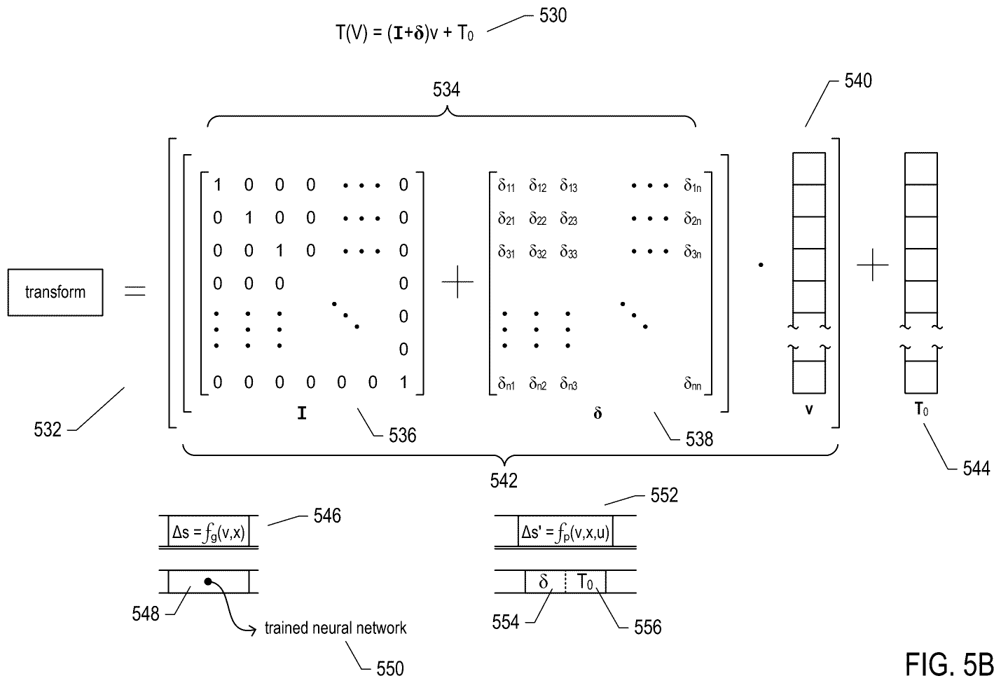

FIGS. 5A-B illustrate one implementation of the deformation process by which a generic efficacy-estimation function is modified to produce a more accurate patient-specific efficacy-estimation function. At the top of FIG. 5A, an implementation of a generic efficacy-estimation function is illustrated in diagram 502. In this implementation, a trained neural network 504, in response to receiving a control-variable vector 506 and a pti vector 508, returns an efficacy estimate 510. Any of many different types of neural networks can be used for a generic efficacy-estimation function, which and can be trained and continuously updated using information contained in the table patient_history in the TD associated with the treatment type and may additionally use, in certain cases, information contained in the patient_history tables of other TDs associated with other, similar treatment types. Diagram 512 in the lower portion of FIG. 5A illustrates one implementation of a patient-specific efficacy-estimation function generated by modifying a generic efficacy-estimation function. The deformation, or modification, of the generic efficacy-estimation function is accomplished via a transform 514 of the input control-variable vector 516 to produce a transformed control-variable vector 518. The transformed control-variable vector is input, along with the pti vector 520 generated for a specific patient, to the trained neural network 504 that represents the generic efficacy-estimation function to produce an initial efficacy estimate 522 to which a constant c 524 is added in order to produce the efficacy-estimation result 526 of the modified or deformed efficacy-estimation function. Thus, rather than attempting to retrain the neural network or begin to train a newly initialized neural network with relatively little, if any, training data, the deformation process instead transforms the input control-variable vector 516 and adds a vertical-adjustment constant c 524 to the output of the neural-network representing the generic efficacy-estimation function. Furthermore, the generic efficacy-estimation-function neural-network is continuously updated by the currently disclosed methods and systems so that it too is improved over time.

FIG. 5B illustrates one implementation of the transform (514 in FIG. 5A) used in the deformation or modification of the generic efficacy-estimation function. A matrix expression 530 for the transform is shown at the top of FIG. 5B. The transform is shown diagrammatically in the middle portion 532 of FIG. 5B. A matrix 534, obtained by adding the identity matrix 530 to a deformation matrix 538, multiplies a control-variable vector 542 to produce a resultant transformed vector 542. A constant transformation vector 544 is added to the resultant transformed vector to produce the final modified control-variable vector (518 in FIG. 5A). Thus, in FIG. 3, the field of a table that contains a reference to a generic efficacy-estimation function 546 contains a reference 548 to a trained neural network 550. A field of a table that contains a patient-specific efficacy-estimation function 552 contains either a deformation matrix 554 and a transformation vector 556 or references to a deformation matrix and transformation vector stored in another table or database.

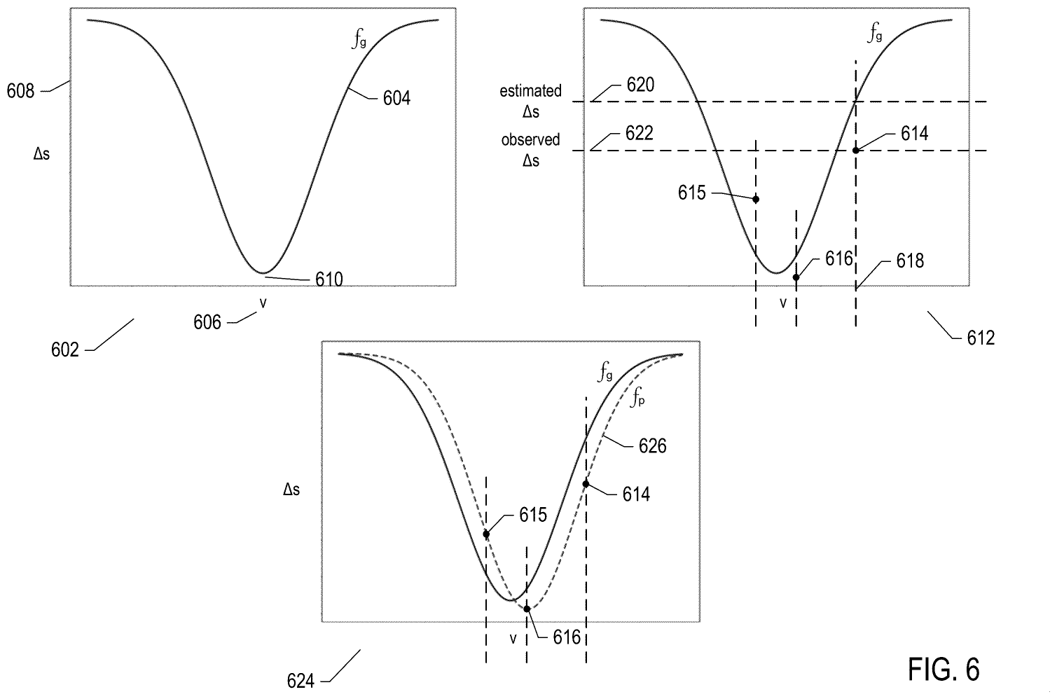

FIG. 6 illustrates, using a 1-dimensional example, the deformation or modification of a generic efficacy-estimation function to produce a patient-specific efficacy-estimation function. A first plot 602 shows the generic efficacy-estimation-function curve 604 for a single control variable plotted with respect to a horizontal axis 606, with the efficacy estimate 608 plotted with respect to a vertical axis. In this simple example, the optimal value for the single control variable lies at the bottom 610 of the well-shaped efficacy-estimation-function curve. In a second plot 612, three experimentally derived data points for a particular patient 614-616 are plotted along with the generic efficacy-estimation-function curve. In other words, for example, for a control-variable value of 618, the generic efficacy-estimation function estimates an efficacy of 620 but an experimental treatment or therapy corresponding to the control-variable value of 618 produces a different observed efficacy 622. The transform discussed above with reference to FIG. 5B is then used, as illustrated in plot 624, to shift, deform, and align the patient-specific efficacy-estimation-function curve 626 with the experimentally derived data points 614-616. Thus, the transformation of the control variable produces a slightly modified or deformed patient-specific efficacy-estimation function that retains much of the information contained in the generic efficacy-estimation function from which it is produced. Simply trying to fit an arbitrary curve through a handful of experimentally derived data points, without the benefit of a generic efficacy-estimation function, would not be possible or, perhaps stated more accurately, would not sufficiently constrain the form of the curve to produce a patient-specific efficacy-estimation function that would be accurate over a reasonable range of possible control-variable vectors. The deformation retains a great deal of knowledge accumulated over many treatments of many different patients while adjusting the generic efficacy-estimation function to create a patient-specific efficacy-estimation function for a particular patient.

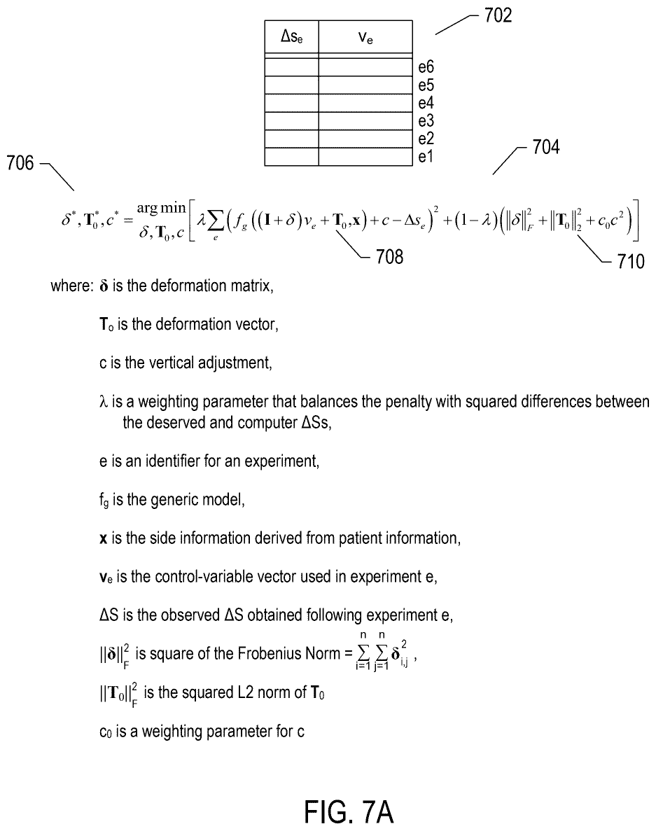

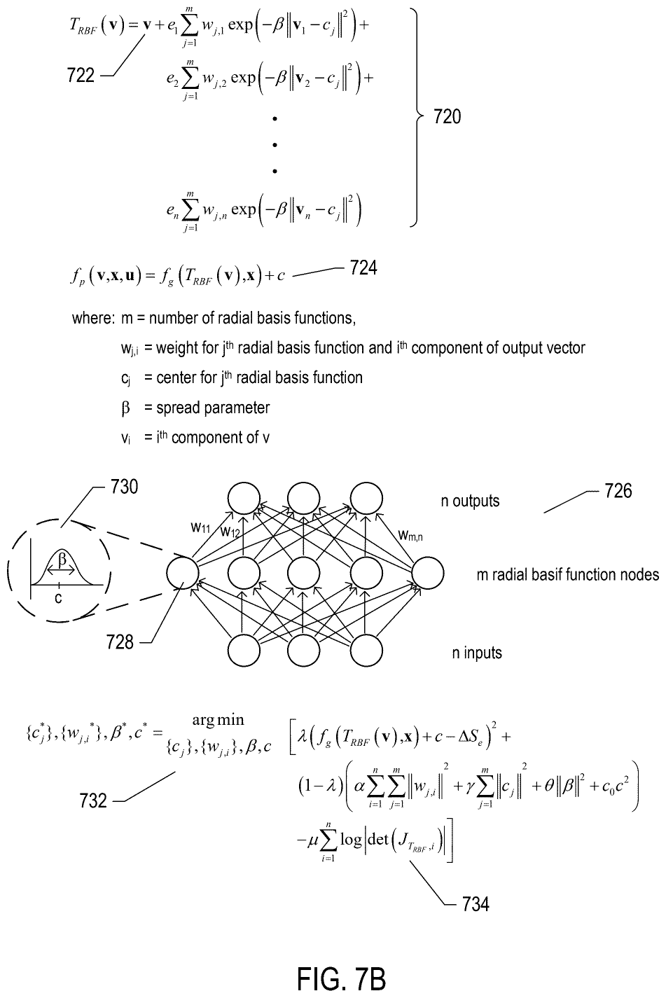

FIGS. 7A-B illustrate the deformation process introduced above with reference to FIG. 6. The process is illustrated in FIG. 7A and uses a table of ΔS/v pairs 702 that are stored in the set vs_pairs in the implementation of the routine “experiment” shown in FIG. 4D and discussed above. The deformation process minimizes the bracketed value 704 over possible values of the deformation matrix δ, transformation vector T₀, and vertical-alignment constant c, as indicated in expressions 706. A first term 708 in the bracketed expression 704 is the sum of the squared differences between the estimated efficacies of the patient-specific efficacy-estimation function parameterized by particular values of the deformation matrix δ, transformation vector T₀, and vertical-alignment constant c and the experimentally observed efficacies and the second term 710 is a penalty term that penalizes large-magnitude deformation matrices 8, transformation vectors T₀, and vertical-alignment constants c. Thus, the minimization of the value represented by the bracketed expression conceptually represents a search for an optimal deformation matrix δ*, transformation vector T₀*, and vertical-alignment constant c* that minimizes the sum of the squared differences between the efficacy estimates generated by the patient-specific efficacy-estimation function parameterized by the optimal deformation matrix δ*, transformation vector T₀*, and vertical-alignment constant c* and the experimentally determined efficacies while, at the same time, constraining the optimal deformation matrix δ*, transformation vector T₀*, and vertical-alignment constant c* by using the penalty term to avoid larger-than-desirable changes to the generic efficacy-estimation function. The penalty term increases in magnitude with increase in the magnitudes of the deformation matrix δ*, transformation vector T₀*, and vertical-alignment constant c* to penalize larger deformations. This penalty-term-constrained minimization seeks an accurate patient-specific efficacy-estimation function that does not differ too greatly from the generic efficacy-estimation function. Any of many standard constrained optimization/minimization techniques can be employed to generate the patient-specific efficacy-estimation function from a table of experimentally derived ΔS/v pairs and an existing generic efficacy-estimation function.

FIG. 7B illustrates an alternative transformation of the input vector for deformation of a generic efficacy-estimation function to that discussed above with reference to FIGS. 5A-B. The alternative transformation uses a radial-basis-function-network transformation described by expression 720 at the top of FIG. 7B. In this expression, the values of the components of the input vector 722 are altered by the addition of values computed from the radial-basis-function network, with e₁, e₂, . . . , eₙ representing the orthonormal basis vectors of control-variable vectors. Expression 724 represents the transformation of a generic efficacy-estimation function to a patient-specific efficacy-estimation function in similar fashion to expression 530 in FIG. 5B. The radial-basis-function network can be viewed as a neural network 726 with each hidden node, such as hidden node 728 representing a radial basis function 730 with a specific center c and spread β. Gaussian-like functions are commonly used as radial-basis functions. Determination of a patient-specific efficacy-estimation function is also a constrained optimization/minimization, as indicated by expression 732 in FIG. 7B, as is the case for the constrained optimization/minimization discussed above with reference to FIG. 7A. In certain implementations, an additional penalty term 734 is included in the bracketed expression for the value that is minimized. This additional penalty term attempts to force the patient-specific efficacy-estimation function towards continuous differentiability.

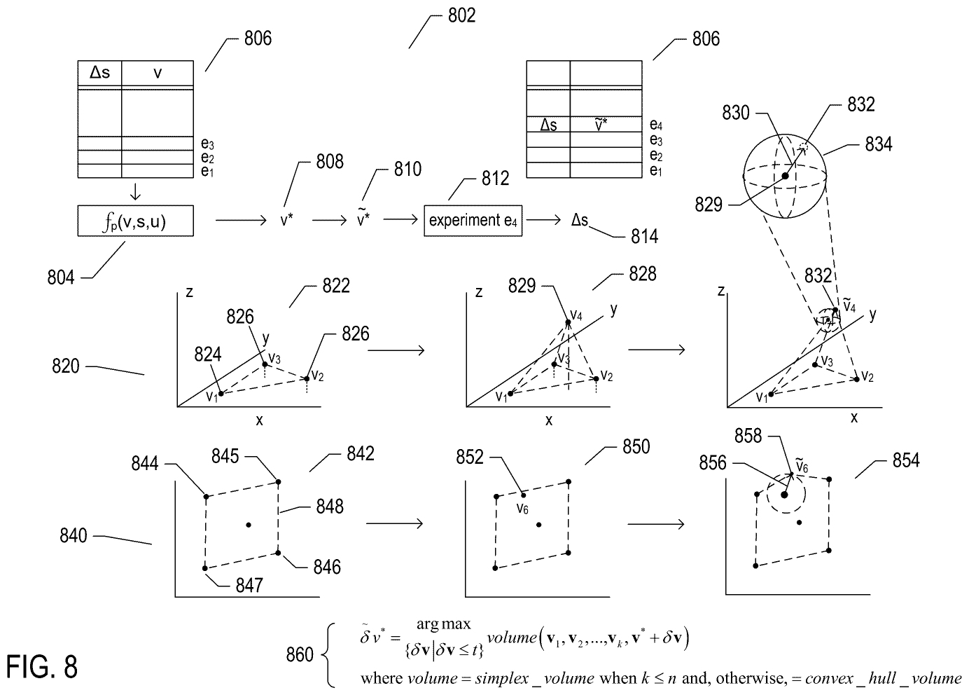

FIG. 8 illustrates a technique used, in certain implementations of the currently disclosed methods and systems, to expand the search space of control-variable vectors explored in the constrained optimization/minimization processes discussed above with reference to FIGS. 7A-B. This process is illustrated in a first diagram 802 the top of FIG. 8. As discussed above, a constrained optimization/minimization process is used to generate a patient-specific efficacy-estimation function 804 from a table of experimentally derived ΔS/v pairs 806. The patient-specific efficacy-estimation function is then used to generate a new control-variable vector 808, or treatment plan, for a next experiment. Rather than use this treatment plan, the search-space expansion technique modifies the new treatment plan to create a modified treatment plan 810 that is then used in a next experiment 812 to generate a new observed result 814 which is added to the table of ΔS/v values 806 along with the modified treatment plan 814. The generation of the modified control-vector is illustrated in two sets of diagrams 820 and 840 in a middle and lower portion of FIG. 8, respectively. In 3-dimensional plot 822, points 824-826 represent the current control vectors in the table of ΔS/v values 806. Plot 828 illustrates addition of a next control-variable vector 829 to the collection of control-variable vectors stored in the table, with control-variable vectors represented by points in a 3-dimensional space, implying that the control-variable vectors each have three elements. However, in order to expand the search space, rather than adding the new control-variable vector 829, a small displacement vector 830 is generated and added to the initial next control-variable vector 829 to produce the modified control-variable vector 832 which is added to the table of ΔS/v values instead of the initial next control-variable vector 829. In the case that the number of control-variable vectors in the table, including the newly added control-variable vector, can be viewed as representing the vertices of a simplex, such as a triangle or tetrahedron in a 3-dimensional or lower-dimensional space, the displacement vector 830 is determined as a displacement vector, equal to or less than a fixed radius of a sphere 834, that generates the greatest resulting area or volume for the simplex. In 2-dimensional plot 842, five 2-dimensional control-variable vectors 844-847 have already been entered into the table of ΔS/v values. Dashed rectangle 848 represents the 2-dimensional convex hull of these 5 points. As shown in plot 850, a next control-variable vector to be added to the table represented by point 852 falls within the convex hull. However, as shown in plot 854, a displacement vector 856 can be generated for the new control-variable vector to modify the new control-variable vector 858 such that the convex hull is expanded in area. Thus, generating a modified next control-variable vector is a constrained optimization/maximization process as indicated by expressions 860 at the bottom of FIG. 8, which is valid for a control-variable-vector of any dimension.

In the following discussion, an implementation for generating one type of severity function, implemented as a trained convolutional neural network, is discussed. This example severity function receives, as input, a sample of a multi-channel sensor output, such as an electroencephalogram (“EEG”) signal, and classifies the sample as being associated with one of multiple severity levels. In the following discussion, the convolutional neural network is described as well as the preparation of inputs to the convolutional neural network. The discussion describes both training of the convolutional neural network as well as use of the convolutional neural network to classify samples selected from the multi-channel-sensor output.

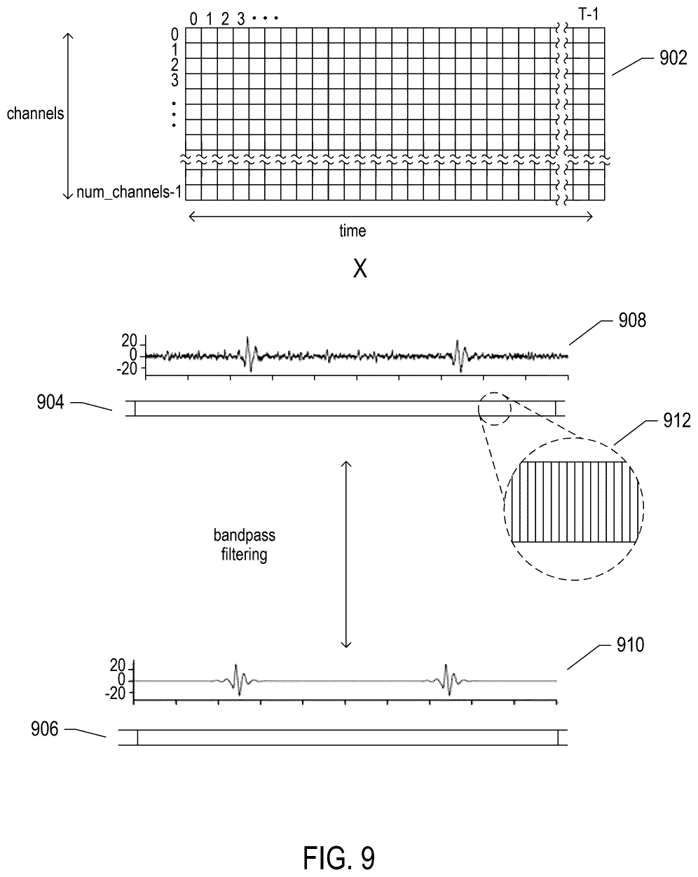

FIG. 9 illustrates initial steps in batch or sample preparation. The multi-channel-sensor signal can be viewed as a 2-dimensional matrix 902 in which each row represents a channel and each column represents a time point. In one initial step, bandpass filtering is applied to each channel or signal component 904 to produce a bandpass-filtered signal component 906. In FIG. 9, the raw signal component is plotted in a 2-dimensional plot 908 and the bandpass-filtered signal component is plotted in a 2-dimensional plot 910 to illustrate the effects of bandpass filtering. Bandpass filtering can be carried out using convolution of Fourier transforms and by other means and selects a specific range of frequencies for the output bandpass-filtered signal. As indicated by inset 912, a signal component may include many time points separated by very short time intervals to provide sufficient resolution.

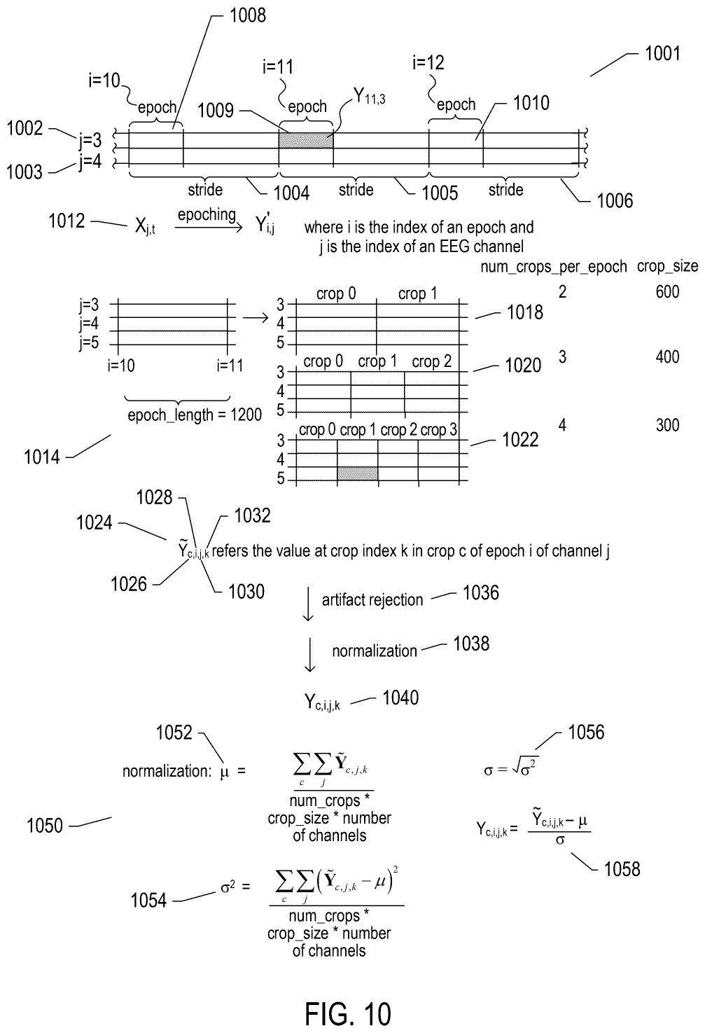

FIG. 10 illustrates additional initial processing steps used to generate signal samples and batches. As shown at the top of FIG. 10 in diagram 1002, the signal is partitioned into multiple contiguous partitions. Diagram 1002 shows several contiguous channels 1002-1003 of a relatively short section of a longer signal that is partitioned into the three partitions 1004-1006. The length of the partitions is specified by a parameter stride. Each partition includes an initial portion referred to as an “epoch.” The signal section shown in diagram 1002 includes the three epochs 1008-1010. Each epoch, which spans all channels, is indexed by an index i and each channel is indexed by an index j, so that the epochs extracted from the signal can be represented as a 2-dimensional tensor with indexes i and j, as indicated in expression 1012. Epochs can be further divided into crops, as indicated in diagram 1014. In the example shown in diagram 1014, an epoch of length 1200 time steps 1016 can be partitioned into two crops, each of length 600 time steps 1018, three crops, each of length 400 time steps 1020, and four crops, each of length 300 time steps 1022. Additional partitionings can be obtained with crop lengths of different sizes. Partitioning into crops transforms a 2-dimensional tensor into a 4-dimensional tensor 1024, with a first index c indicating a particular crop within an epoch 1026, a second index i indicating a particular epoch 1028, a third index j indicating a particular channel 1030, and a fourth index k indicating a particular time step within the crop 1032. Artifact rejection 1036 and normalization 1038 are then applied to the bandpass filtered, epoched, and cropped signal to generate a normalized signal 1040. Artifact rejection involves recognizing spurious features in the signal and eliminating them, such as sharp signal changes due to irrelevant environmental changes, irrelevant physiological changes of the patient, instrument or device noise, and other such phenomena. The normalization method is represented by equations 1050 at the bottom of FIG. 10. This type of normalization involves computation of a mean 1052 and variance 1054 for all of the data points in the channels, computing a standard deviation for the data points 1056, and then subtracting the mean from each data point and dividing the result by the standard deviation 1058. Normalization may be alternatively carried out on a per-channel basis.

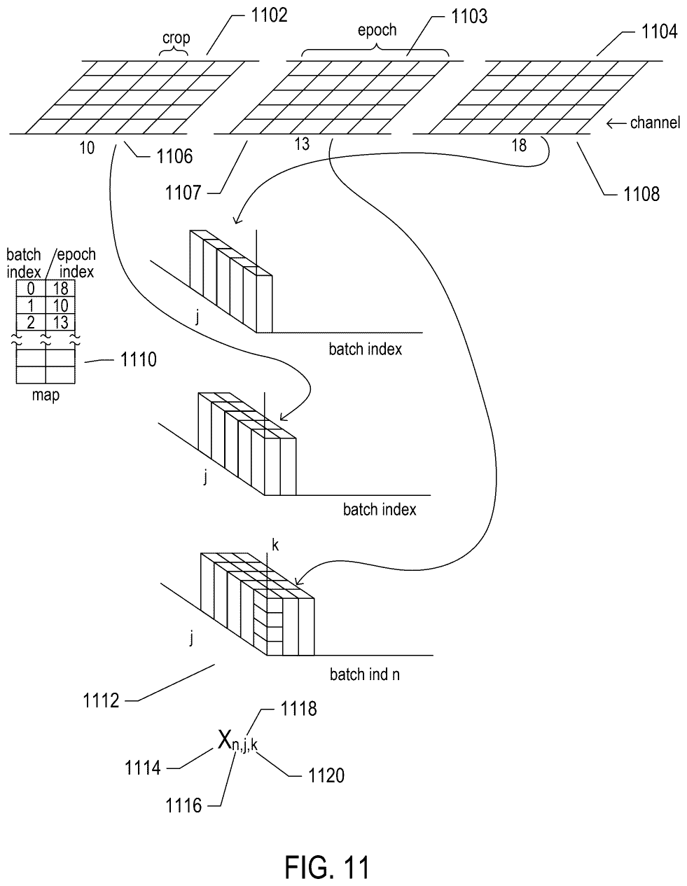

FIG. 11 illustrates generation of a batch, for training, or a sample, for classification. The batches or samples generated by selecting crops, such as crops 1102-1104, from epochs, such as epochs 1106-1108, in an order specified in a map 1110. The selected crops are assembled in order to generate the batch or sample as a 3-dimensional matrix or tensor 1112. This tensor 1114 includes a first index indicating a crop within the batch 1116, a second index indicating a channel within the signal 1118, and a third index representing a particular data point within a batch and channel 1120.

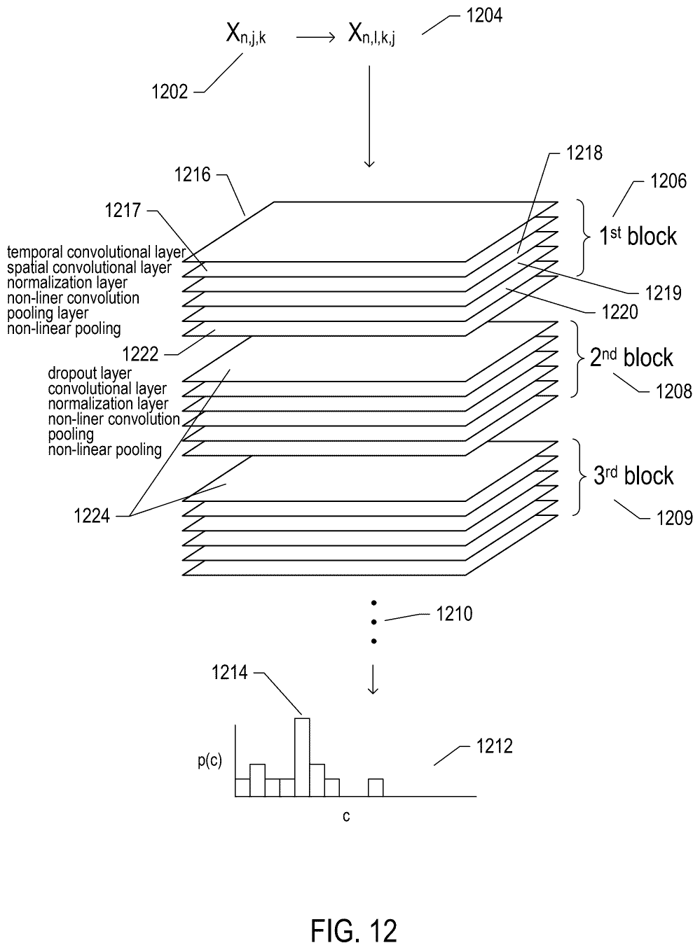

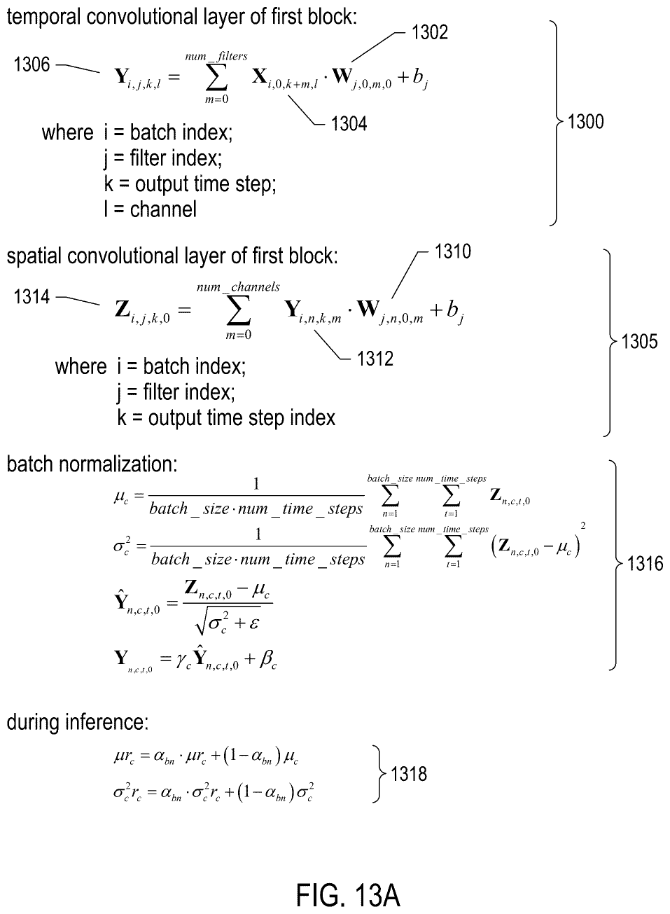

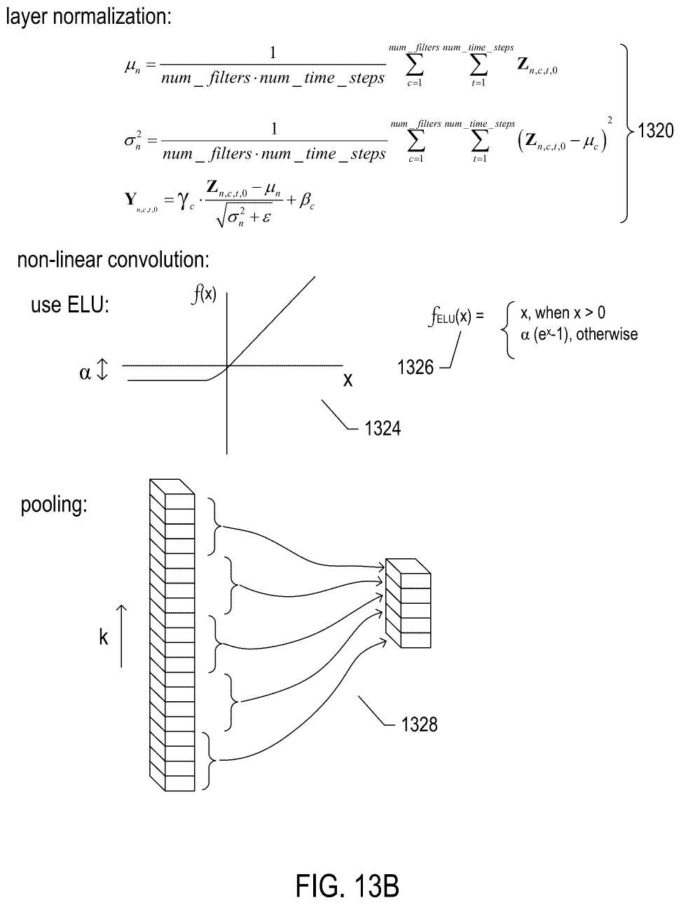

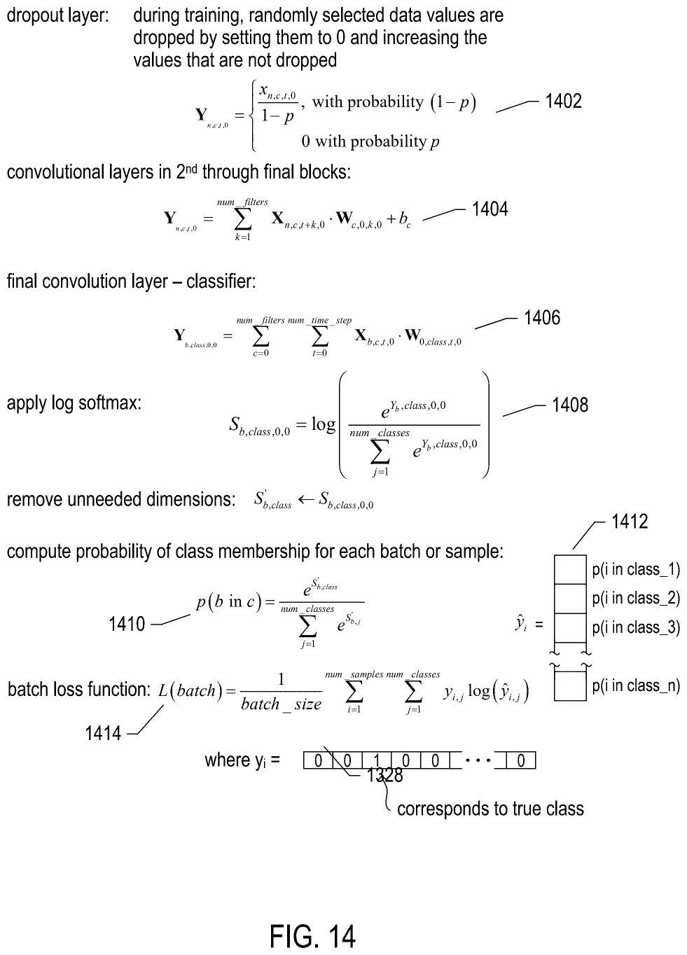

FIG. 12 illustrates one implementation of the convolutional neural network that implements the example severity function disclosed in the current document. A batch or sample 1202 is expanded to four dimensions 1204 by introducing a new singleton dimension as the second dimension so that certain already existing convolutional neural networks that expect 4-dimensional-tensor inputs can be employed. The convolutional neural network used in the described implementation includes a first block of layers 1206 and one or more additional blocks 1208-1209, with ellipsis 1210 indicating the possibility of additional blocks. The output of the convolutional neural network is a probability distribution 1212 that indicates the probabilities of the sample or batch having each of the various different possible categories. A category with greatest probability 1214 can be selected as the category associated with the sample or batch. The first block of layers includes a temporal convolution layer 1216, a spatial convolutional layer 1217 a normalization layer 1218 a non-linear convolution layer 1219 a pooling layer 1220 and a non-linear pooling layer 1222 each successive block includes similar layers as well as a first dropout layer 1224. These layers are briefly described in FIGS. 13A-B and 14.

FIG. 13A briefly describes the temporal, spatial, and batch-normalization layers. The temporal convolutional layer 1300 applies a set of filters 1302 to an input batch or sample 1304 to generate an output 1306. The range of time steps in the output is decreased by this process. The spatial convolutional layer 1308 applies a number of filters 1310 to the input 1312 to generate an output 1314. The batch-normalization layer carries out a normalization via a computed batch mean and variance, as indicated by expressions 1316 during training and, when the convolutional neural network is used to classify samples, uses a running mean and a running variance that are updated during the normalization process, as indicated by expressions 1318, where the parameter α_{bn} represents a momentum or learning rate. Turning to FIG. 13B, the layer normalization layer carries out a normalization indicated by expressions 1320. The non-linear convolution layer employs the ELU activation function plotted in plot 1324 and defined in expression 1326. The pooling layer compresses a sample or batch along the temporal or time-step dimension, as indicated by diagram 1328. The value used to represent a set of contiguous data points in the time-step dimension may be the maximum value of a data point in the set of contiguous data points, a mean value of the data points in the set of continuous data points, or another type of computed value.

Turning to FIG. 14, the dropout layer in each of the second through final blocks of the convolutional neural network randomly sets various data points to 0, as indicated by expression 1402, and is used only during training. The convolutional layers used in the second through final. blocks are described by expression 1404. The convolutional neural network includes a final convolutional layer or classifier layer described by expression 1406. The classifier layer generates a value for each different class using the log softmax function, as indicated by expression 1408. As indicated by expression 1410, these values can be used to generate a probability distribution 1412 (1212 in FIG. 12) that can be used to assign a class to a sample or batch. Finally, a cross-entropy loss function 1414 is used during training of the convolutional network.

### Computer Hardware, Complex Computational Systems, and Operating Systems and Virtualization

The term “abstraction” is not, in any way, intended to mean or suggest an abstract idea or concept. Computational abstractions are tangible, physical interfaces that are implemented, ultimately, using physical computer hardware, data-storage devices, and communications systems. Instead, the term “abstraction” refers, in the current discussion, to a logical level of functionality encapsulated within one or more concrete, tangible, physically implemented computer systems with defined interfaces through which electronically encoded data is exchanged, process execution launched, and electronic services are provided. Interfaces may include graphical and textual data displayed on physical display devices as well as computer programs and routines that control physical computer processors to carry out various tasks and operations and that are invoked through electronically implemented application programming interfaces (“APIs”) and other electronically implemented interfaces. There is a tendency among those unfamiliar with modern technology and science to misinterpret the terms “abstract” and “abstraction,” when used to describe certain aspects of modern computing. For example, one frequently encounters assertions that, because a computational system is described in terms of abstractions, functional layers, and interfaces, the computational system is somehow different from a physical machine or device. Such allegations are unfounded. One only needs to disconnect a computer system or group of computer systems from their respective power supplies to appreciate the physical, machine nature of complex computer technologies. One also frequently encounters statements that characterize a computational technology as being “only software,” and thus not a machine or device. Software is essentially a sequence of encoded symbols, such as a printout of a computer program or digitally encoded computer instructions sequentially stored in a file on an optical disk or within an electromechanical mass-storage device. Software alone can do nothing. It is only when encoded computer instructions are loaded into an electronic memory within a computer system and executed on a physical processor that so-called “software implemented” functionality is provided. The digitally encoded computer instructions are an essential and physical control component of processor-controlled machines and devices, no less essential and physical than a cam-shaft control system in an internal-combustion engine. Multi-cloud aggregations, cloud-computing services, virtual-machine containers and virtual machines, communications interfaces, and many of the other entities are tangible, physical components of physical, electro-optical-mechanical. computer systems.

FIG. 15 provides a general architectural diagram for various types of computers. The computer system contains one or multiple central processing units (“CPUs”) 1502-1505, one or more electronic memories 1508 interconnected with the CPUs by a CPU/memory-subsystem bus 1510 or multiple buses, a first bridge 1512 that interconnects the CPU/memory-subsystem bus 1510 with additional buses 1514 and 1516, or other types of high-speed interconnection media, including multiple, high-speed serial interconnects. These buses or serial interconnections, in turn, connect the CPUs and memory with specialized processors, such as a graphics processor 1518, and with one or more additional bridges 1520, which are interconnected with high-speed serial links or with multiple controllers 1522-1527, such as controller 1527, that provide access to various different types of mass-storage devices 1528, electronic displays, input devices, and other such components, subcomponents, and computational resources. It should be noted that computer-readable data-storage devices include optical and electromagnetic disks, electronic memories, and other physical data-storage devices. Those familiar with modern science and technology appreciate that electromagnetic radiation and propagating signals do not store data for subsequent retrieval and can transiently “store” only a byte or less of information per mile, far less information than needed to encode even the simplest of routines.

Of course, there are many different types of computer-system architectures that differ from one another in the number of different memories, including different types of hierarchical cache memories, the number of processors and the connectivity of the processors with other system components, the number of internal communications buses and serial links, and in many other ways. However, computer systems generally execute stored programs by fetching instructions from memory and executing the instructions in one or more processors. Computer systems include general-purpose computer systems, such as personal computers (“PCs”), various types of servers and workstations, and higher-end mainframe computers, but may also include a plethora of various types of special-purpose computing devices, including data-storage systems, communications routers, network nodes, tablet computers, and mobile telephones.

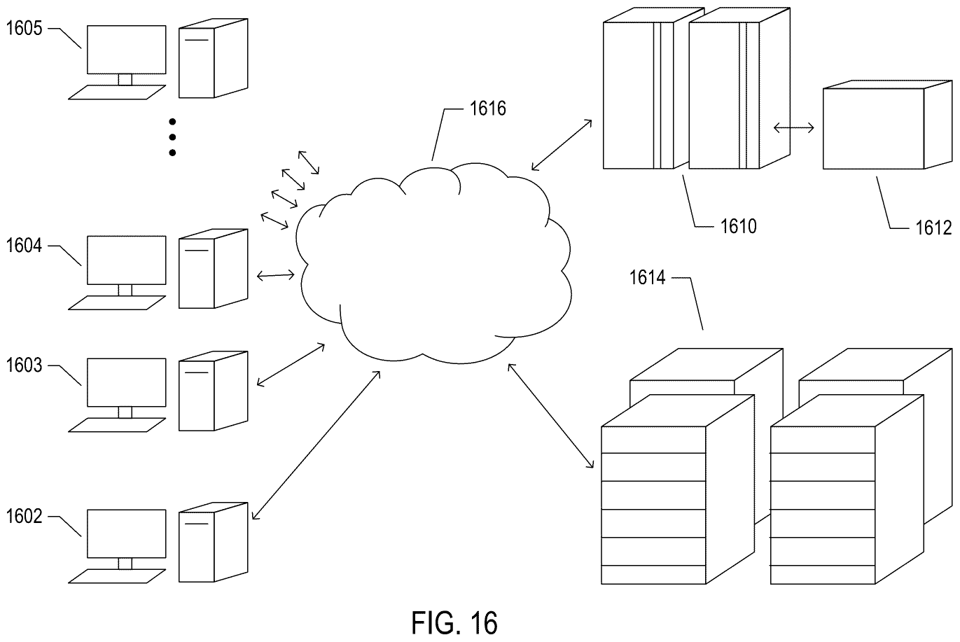

FIG. 16 illustrates an Internet-connected distributed computer system. As communications and networking technologies have evolved in capability and accessibility, and as the computational bandwidths, data-storage capacities, and other capabilities and capacities of various types of computer systems have steadily and rapidly increased, much of modern computing now generally involves large distributed systems and computers interconnected by local networks, wide-area networks, wireless communications, and the Internet. FIG. 16 shows a typical distributed system in which a large number of PCs 1602-1605, a high-end distributed mainframe system 1610 with a large data-storage system 1612, and a large computer center 1614 with large numbers of rack-mounted servers or blade servers all interconnected through various communications and networking systems that together comprise the Internet 1616. Such distributed computer systems provide diverse arrays of functionalities. For example, a PC user sitting in a home office may access hundreds of millions of different web sites provided by hundreds of thousands of different web servers throughout the world and may access high-computational-bandwidth computing services from remote computer facilities for running complex computational tasks.

Until recently, computational services were generally provided by computer systems and data centers purchased, configured, managed, and maintained by service-provider organizations. For example, an e-commerce retailer generally purchased, configured, managed, and maintained a data center including numerous web servers, back-end computer systems, and data-storage systems for serving web pages to remote customers, receiving orders through the web-page interface, processing the orders, tracking completed orders, and other myriad different tasks associated with an e-commerce enterprise.

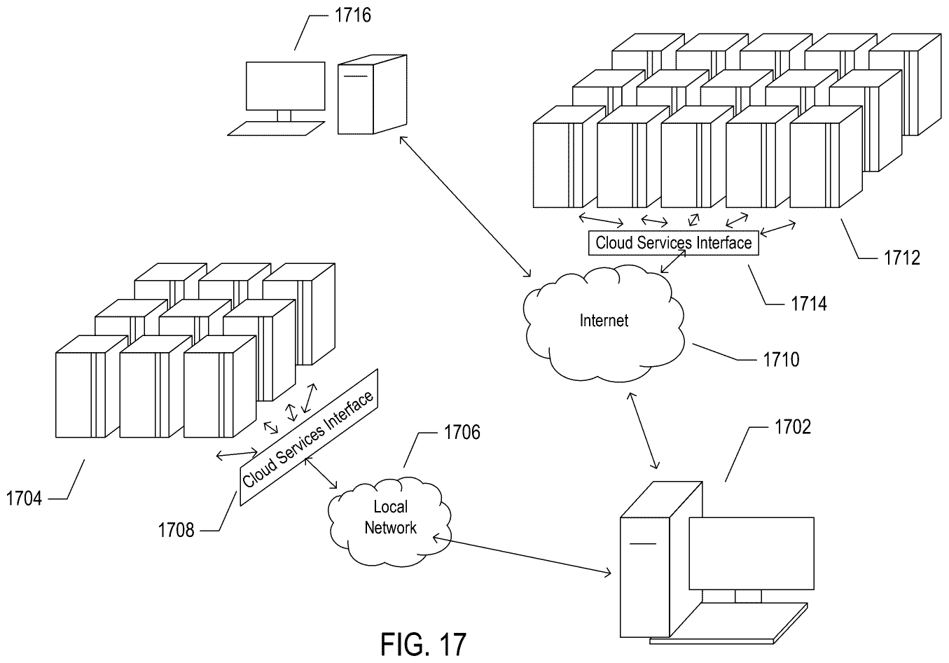

FIG. 17 illustrates cloud computing. In the recently developed cloud-computing paradigm, computing cycles and data-storage facilities are provided to organizations and individuals by cloud-computing providers. In addition, larger organizations may elect to establish private cloud-computing facilities in addition to, or instead of, subscribing to computing services provided by public cloud-computing service providers. In FIG. 17, a system administrator for an organization, using a PC 1702, accesses the organization's private cloud 1704 through a local network 1706 and private-cloud interface 1708 and also accesses, through the Internet 1710, a public cloud 1712 through a public-cloud services interface 1714. The administrator can, in either the case of the private cloud 1704 or public cloud 1712, configure virtual computer systems and even entire virtual data centers and launch execution of application programs on the virtual computer systems and virtual data centers in order to carry out any of many different types of computational tasks. As one example, a small organization may configure and run a virtual data center within a public cloud that executes web servers to provide an e-commerce interface through the public cloud to remote customers of the organization, such as a user viewing the organization's e-commerce web pages on a remote user system 1716.

Cloud-computing facilities are intended to provide computational bandwidth and data-storage services much as utility companies provide electrical power and water to consumers. Cloud computing provides enormous advantages to small organizations without the resources to purchase, manage, and maintain in-house data centers. Such organizations can dynamically add and delete virtual computer systems from their virtual data centers within public clouds in order to track computational-bandwidth and data-storage needs, rather than purchasing sufficient computer systems within a physical data center to handle peak computational-bandwidth and data-storage demands. Moreover, small organizations can completely avoid the overhead of maintaining and managing physical computer systems, including hiring and periodically retraining information-technology specialists and continuously paying for operating-system and database-management-system upgrades. Furthermore, cloud-computing interfaces allow for easy and straightforward configuration of virtual computing facilities, flexibility in the types of applications and operating systems that can be configured, and other functionalities that are useful even for owners and administrators of private cloud-computing facilities used by a single organization.

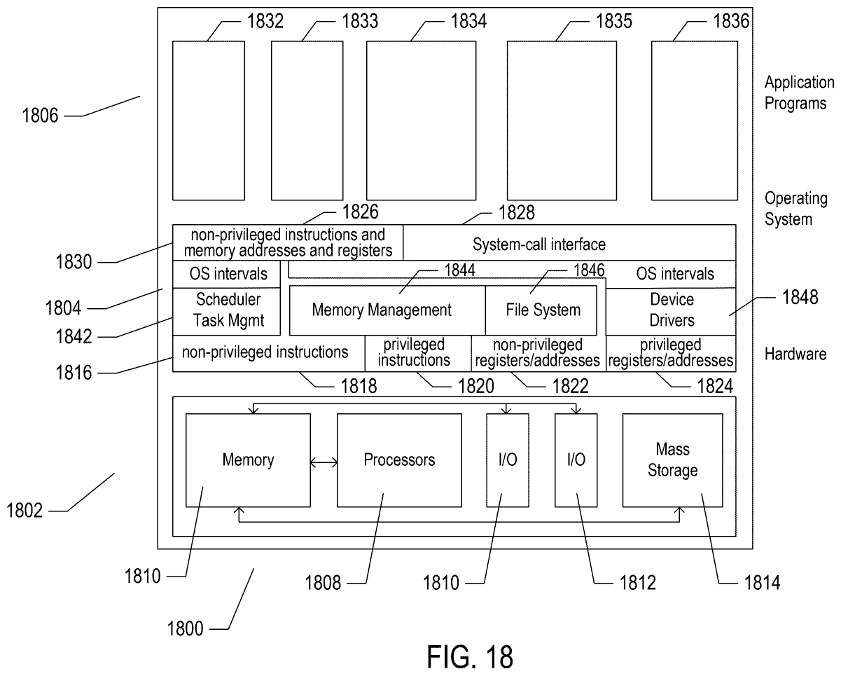

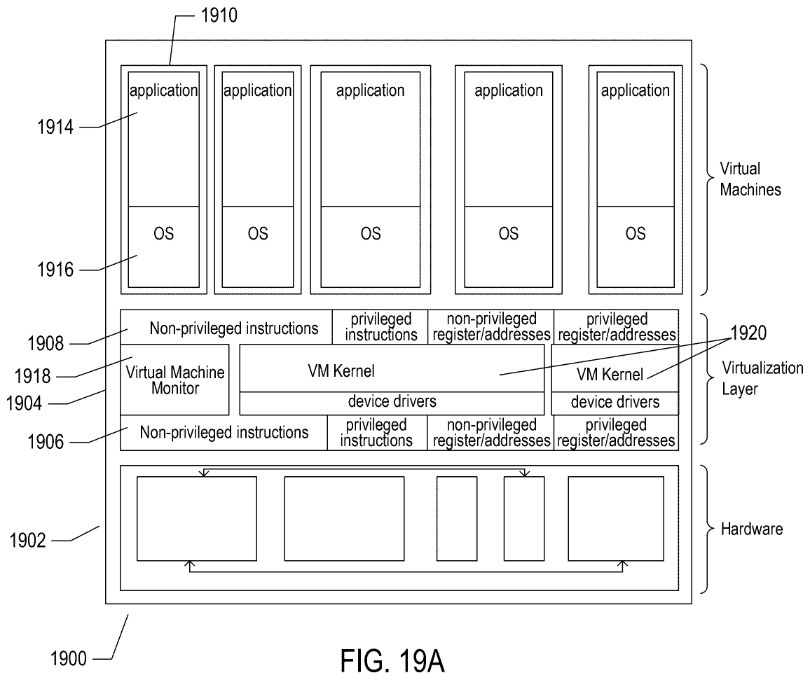

FIG. 18 illustrates generalized hardware and software components of a general-purpose computer system, such as a general-purpose computer system having an architecture similar to that shown in FIG. 1. The computer system 1800 is often considered to include three fundamental layers: (1) a hardware layer or level 1802; (2) an operating-system layer or level 1804; and (3) an application-program layer or level 1806. The hardware layer 1802 includes one or more processors 1808, system memory 1810, various different types of input-output (“I/O”) devices 1810 and 1812, and mass-storage devices 1814. Of course, the hardware level also includes many other components, including power supplies, internal communications links and buses, specialized integrated circuits, many different types of processor-controlled or microprocessor-controlled peripheral devices and controllers, and many other components. The operating system 1804 interfaces to the hardware level 1802 through a low-level operating system and hardware interface 1816 generally comprising a set of non-privileged computer instructions 1818, a set of privileged computer instructions 1820, a set of non-privileged registers and memory addresses 1822, and a set of privileged registers and memory addresses 1824. In general, the operating system exposes non-privileged instructions, non-privileged registers, and non-privileged memory addresses 1826 and a system-call interface 1828 as an operating-system interface 1830 to application programs 1832-1836 that execute within an execution environment provided to the application programs by the operating system. The operating system, alone, accesses the privileged instructions, privileged registers, and privileged memory addresses. By reserving access to privileged instructions, privileged registers, and privileged memory addresses, the operating system can ensure that application programs and other higher-level computational entities cannot interfere with one another's execution and cannot change the overall state of the computer system in ways that could deleteriously impact system operation. The operating system includes many internal components and modules, including a scheduler 1842, memory management 1844, a file system 1846, device drivers 1848, and many other components and modules. To a certain degree, modern operating systems provide numerous levels of abstraction above the hardware level, including virtual memory, which provides to each application program and other computational entities a separate, large, linear memory-address space that is mapped by the operating system to various electronic memories and mass-storage devices. The scheduler orchestrates interleaved execution of various different application programs and higher-level computational entities, providing to each application program a virtual, stand-alone system devoted entirely to the application program. From the application program's standpoint, the application program executes continuously without concern for the need to share processor resources and other system resources with other application programs and higher-level computational entities. The device drivers abstract details of hardware-component operation, allowing application programs to employ the system-call interface for transmitting and receiving data to and from communications networks, mass-storage devices, and other I/O devices and subsystems. The file system 1836 facilitates abstraction of mass-storage-device and memory resources as a high-level, easy-to-access, file-system interface. Thus, the development and evolution of the operating system has resulted in the generation of a type of multi-faceted virtual execution environment for application programs and other higher-level computational entities.

While the execution environments provided by operating systems have proved to be an enormously successful level of abstraction within computer systems, the operating-system-provided level of abstraction is nonetheless associated with difficulties and challenges for developers and users of application programs and other higher-level computational entities. One difficulty arises from the fact that there are many different operating systems that run within various different types of computer hardware. In many cases, popular application programs and computational systems are developed to run on only a subset of the available operating systems and can therefore be executed within only a subset of the various different types of computer systems on which the operating systems are designed to run. Often, even when an application program or other computational system is ported to additional operating systems, the application program or other computational system can nonetheless run more efficiently on the operating systems for which the application program or other computational system was originally targeted. Another difficulty arises from the increasingly distributed nature of computer systems. Although distributed operating systems are the subject of considerable research and development efforts, many of the popular operating systems are designed primarily for execution on a single computer system. In many cases, it is difficult to move application programs, in real time, between the different computer systems of a distributed computer system for high-availability, fault-tolerance, and load-balancing purposes. The problems are even greater in heterogeneous distributed computer systems which include different types of hardware and devices running different types of operating systems. Operating systems continue to evolve, as a result of which certain older application programs and other computational entities may be incompatible with more recent versions of operating systems for which they are targeted, creating compatibility issues that are particularly difficult to manage in large distributed systems.

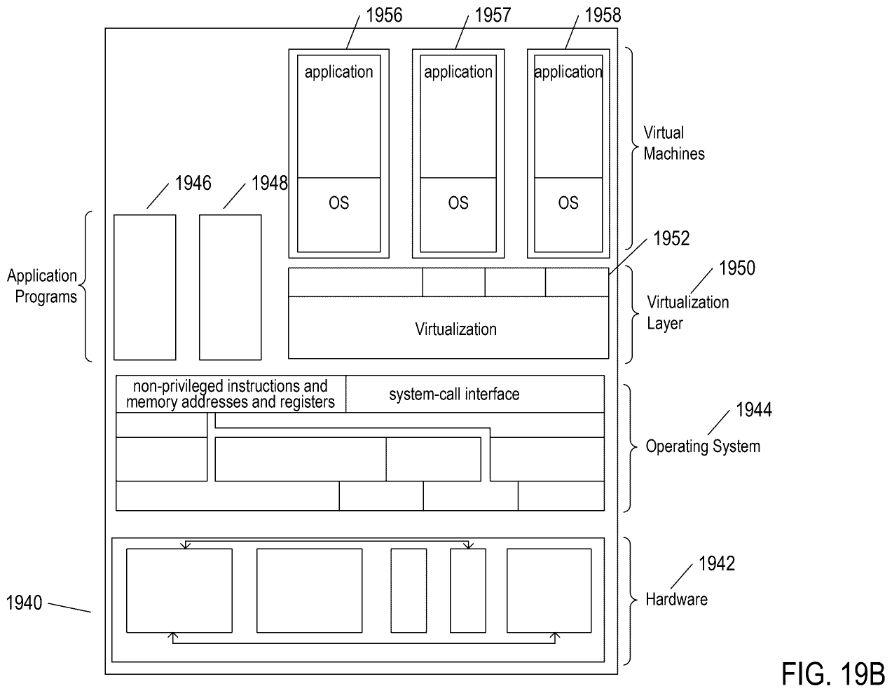

For all of these reasons, a higher level of abstraction, referred to as the “virtual machine,” has been developed and evolved to further abstract computer hardware in order to address many difficulties and challenges associated with traditional computing systems, including the compatibility issues discussed above. FIGS. 19A-B illustrate several types of virtual machine and virtual-machine execution environments. FIGS. 19A-B use the same illustration conventions as used in FIG. 18. FIG. 19A shows a first type of virtualization. The computer system 1900 in FIG. 19A includes the same hardware layer 1902 as the hardware layer 1802 shown in FIG. 18. However, rather than providing an operating system layer directly above the hardware layer, as in FIG. 18, the virtualized computing environment illustrated in FIG. 19A features a virtualization layer 1904 that interfaces through a virtualization-layer/hardware-layer interface 1906, equivalent to interface 1816 in FIG. 18, to the hardware. The virtualization layer provides a hardware-like interface 1908 to a number of virtual machines, such as virtual machine 1910, executing above the virtualization layer in a virtual-machine layer 1912. Each virtual machine includes one or more application programs or other higher-level computational entities packaged together with an operating system, referred to as a “guest operating system,” such as application 1914 and guest operating system 1916 packaged together within virtual machine 1910. Each virtual machine is thus equivalent to the operating-system layer 1804 and application-program layer 1806 in the general-purpose computer system shown in FIG. 18. Each guest operating system within a virtual machine interfaces to the virtualization-layer interface 1908 rather than to the actual hardware interface 1906. The virtualization layer partitions hardware resources into abstract virtual-hardware layers to which each guest operating system within a virtual machine interfaces. The guest operating systems within the virtual machines, in general, are unaware of the virtualization layer and operate as if they were directly accessing a true hardware interface. The virtualization layer ensures that each of the virtual machines currently executing within the virtual environment receive a fair allocation of underlying hardware resources and that all virtual machines receive sufficient resources to progress in execution. The virtualization-layer interface 1908 may differ for different guest operating systems. For example, the virtualization layer is generally able to provide virtual hardware interfaces for a variety of different types of computer hardware. This allows, as one example, a virtual machine that includes a guest operating system designed for a particular computer architecture to run on hardware of a different architecture. The number of virtual machines need not be equal to the number of physical processors or even a multiple of the number of processors.

The virtualization layer includes a virtual-machine-monitor module 1918 (“VMM”) that virtualizes physical processors in the hardware layer to create virtual processors on which each of the virtual machines executes. For execution efficiency, the virtualization layer attempts to allow virtual machines to directly execute non-privileged instructions and to directly access non-privileged registers and memory. However, when the guest operating system within a virtual machine accesses virtual privileged instructions, virtual privileged registers, and virtual privileged memory through the virtualization-layer interface 1908, the accesses result in execution of virtualization-layer code to simulate or emulate the privileged resources. The virtualization layer additionally includes a kernel module 1920 that manages memory, communications, and data-storage machine resources on behalf of executing virtual machines (“VM kernel”). The VM kernel, for example, maintains shadow page tables on each virtual machine so that hardware-level virtual-memory facilities can be used to process memory accesses. The VM kernel additionally includes routines that implement virtual communications and data-storage devices as well as device drivers that directly control the operation of underlying hardware communications and data-storage devices. Similarly, the VM kernel virtualizes various other types of I/O devices, including keyboards, optical-disk drives, and other such devices. The virtualization layer essentially schedules execution of virtual machines much like an operating system schedules execution of application programs, so that the virtual machines each execute within a complete and fully functional virtual hardware layer.

FIG. 19B illustrates a second type of virtualization. In FIG. 19B, the computer system 1940 includes the same hardware layer 1942 and software layer 1944 as the hardware layer 402 shown in FIG. 4. Several application programs 1946 and 1948 are shown running in the execution environment provided by the operating system. In addition, a virtualization layer 1950 is also provided, in computer 1940, but, unlike the virtualization layer 1904 discussed with reference to FIG. 19A, virtualization layer 1950 is layered above the operating system 1944, referred to as the “host OS,” and uses the operating system interface to access operating-system-provided functionality as well as the hardware. The virtualization layer 1950 comprises primarily a VMM and a hardware-like interface 1952, similar to hardware-like interface 1908 in FIG. 19A. The virtualization-layer/hardware-layer interface 1952, equivalent to. interface 416 in FIG. 4, provides an execution environment for a number of virtual machines 1956-1958, each including one or more application programs or other higher-level computational entities packaged together with a guest operating system.

### Neural Networks

FIG. 20 illustrates fundamental components of a feed-forward neural network. Expressions 2002 mathematically represent ideal operation of a neural network as a function f(x). The function receives an input vector x and outputs a corresponding output vector y 1103. For example, an input vector may be a digital image represented by a 2-dimensional array of pixel values in an electronic document or may be an ordered set of numeric or alphanumeric values. Similarly, the output vector may be, for example, an altered digital image, an ordered set of one or more numeric or alphanumeric values, an electronic document, or one or more numeric values. The initial expression of expressions 2002 represents the ideal operation of the neural network. In other words, the output vector y represents the ideal, or desired, output for corresponding input vector x. However, in actual operation, a physically implemented neural network \hat{f}(x), as represented by the second expression of expressions 2002, returns a physically generated output vector ŷ that may differ from the ideal or desired output vector y. An output vector produced by the physically implemented neural network is associated with an error or loss value. A common error or loss value is the square of the distance between the two points represented by the ideal output vector y and the output vector produced by the neural network ŷ. The distance between the two points represented by the ideal output vector and the output vector produced by the neural network, with optional scaling, may also be used as the error or loss. A neural network is trained using a training dataset comprising input-vector/ideal-output-vector pairs, generally obtained by human or human-assisted assignment of ideal-output vectors to selected input vectors. The ideal-output vectors in the training dataset are often referred to as “labels.” During training, the error associated with each output vector, produced by the neural network in response to input to the neural network of a training-dataset input vector, is used to adjust internal weights within the neural network in order to minimize the error or loss. Thus, the accuracy and reliability of a trained neural network is highly dependent on the accuracy and completeness of the training dataset.

As shown in the middle portion 2006 of FIG. 20, a feed-forward neural network generally consists of layers of nodes, including an input layer 2008, an output layer 2010, and one or more hidden layers 2012. These layers can be numerically labeled 1, 2, 3, . . . , L−1, L as shown in FIG. 20. In general, the input layer contains a node for each element of the input vector and the output layer contains one node for each element of the output vector. The input layer and/or output layer may each have one or more nodes. In the following discussion, the nodes of a first level with a numeric label lower in value than that of a second layer are referred to as being higher-level nodes with respect to the nodes of the second layer. The input-layer nodes are thus the highest-level nodes. The nodes are interconnected to form a graph, as indicated by line segments, such as line segment 2014.

The lower portion of FIG. 20 (2020 in FIG. 20) illustrates a feed-forward neural-network node. The neural-network node 2022 receives inputs 2024-2027 from one or more next-higher-level nodes and generates an output 2028 that is distributed to one or more next-lower-level nodes 2030. The inputs and outputs are referred to as “activations,” represented by superscripted-and-subscripted symbols “a” in FIG. 20, such as the activation symbol 2024. An input component 2036 within a node collects the input activations and generates a weighted sum of these input activations to which a weighted internal activation a₀is added. An activation component 2038 within the node is represented by a function g( ), referred to as an “activation function,” that is used in an output component 2040 of the node to generate the output activation of the node based on the input collected by the input component 2036. The neural-network node 2022 represents a generic hidden-layer node. Input-layer nodes lack the input component 2036 and each receive a single input value representing an element of an input vector. Output-component nodes output a single value representing an element of the output vector. The values of the weights used to generate the cumulative input by the input component 2036 are determined by training, as previously mentioned. In general, the input, outputs, and activation function are predetermined and constant, although, in certain types of neural networks, these may also be at least partly adjustable parameters. In FIG. 20, three different possible activation functions are indicated by expressions 2042-2044. The first expression is a binary activation function and the third expression represents a sigmoidal relationship between input and output that is commonly used in neural networks and other types of machine-learning systems, both functions producing an activation in the range [0, 1]. The second function is also sigmoidal, but produces an activation in the range [−1, 1].

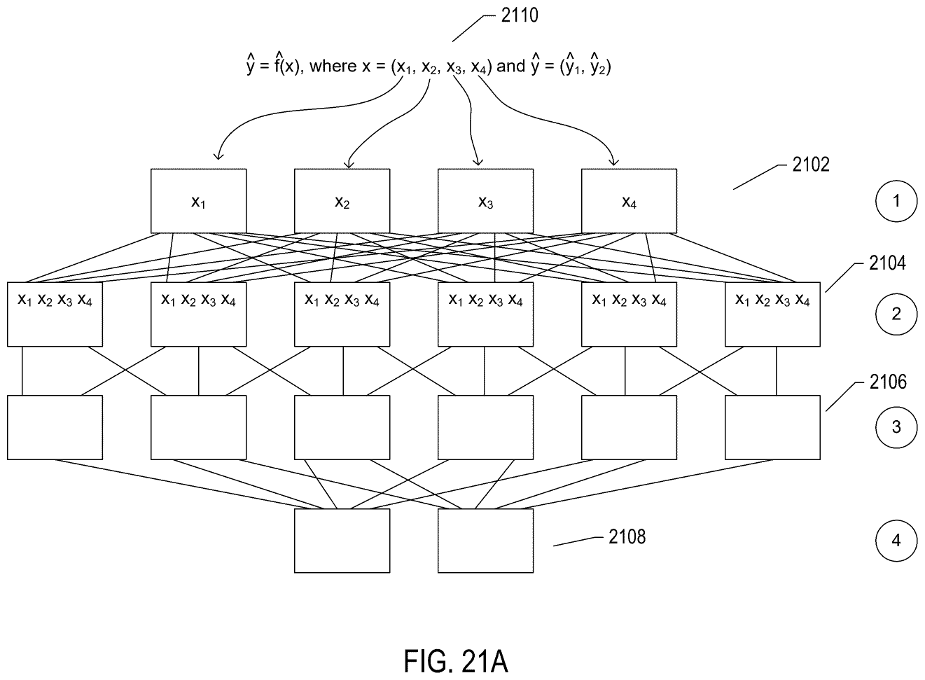

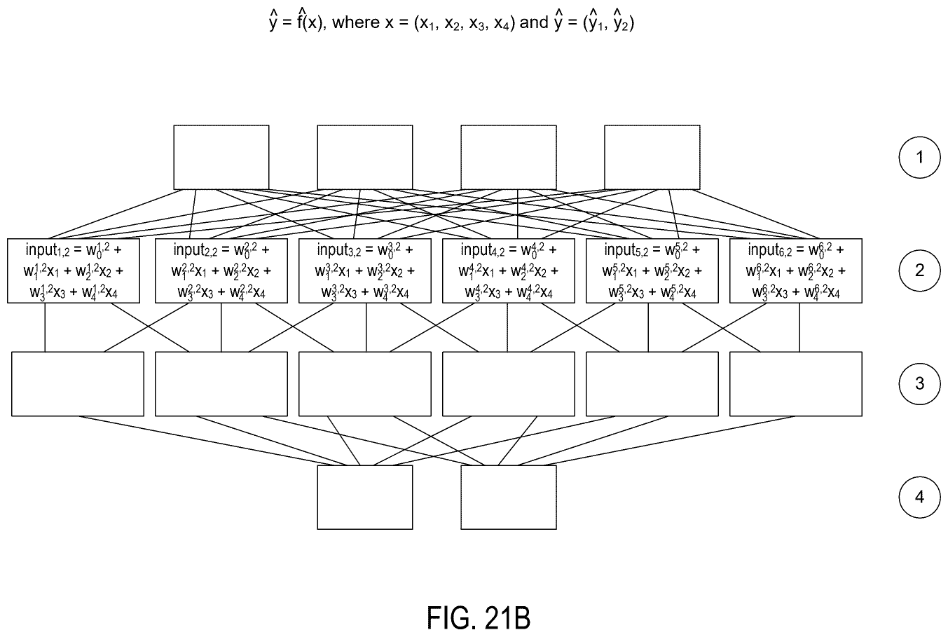

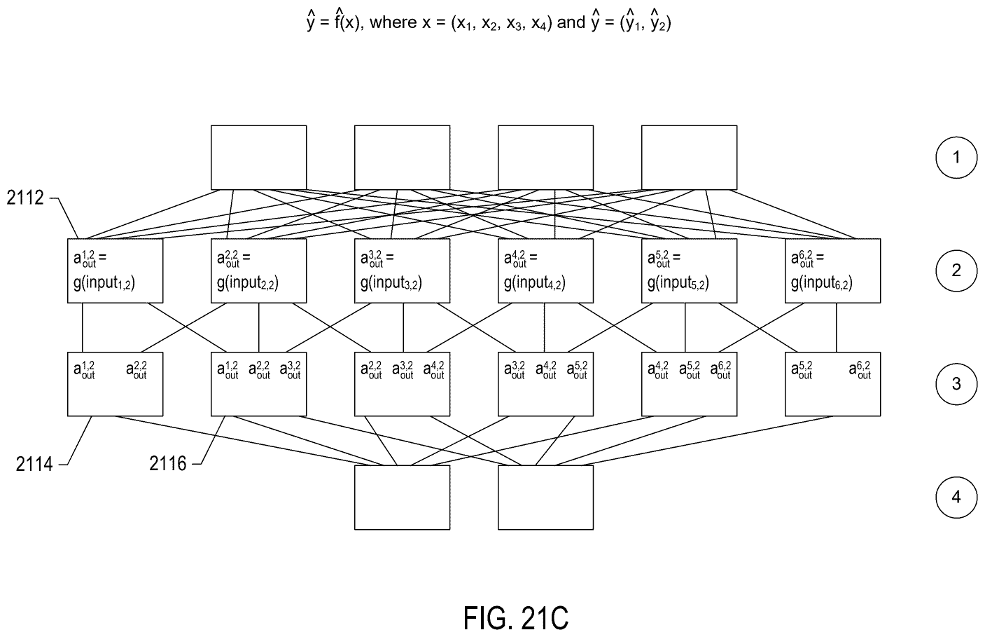

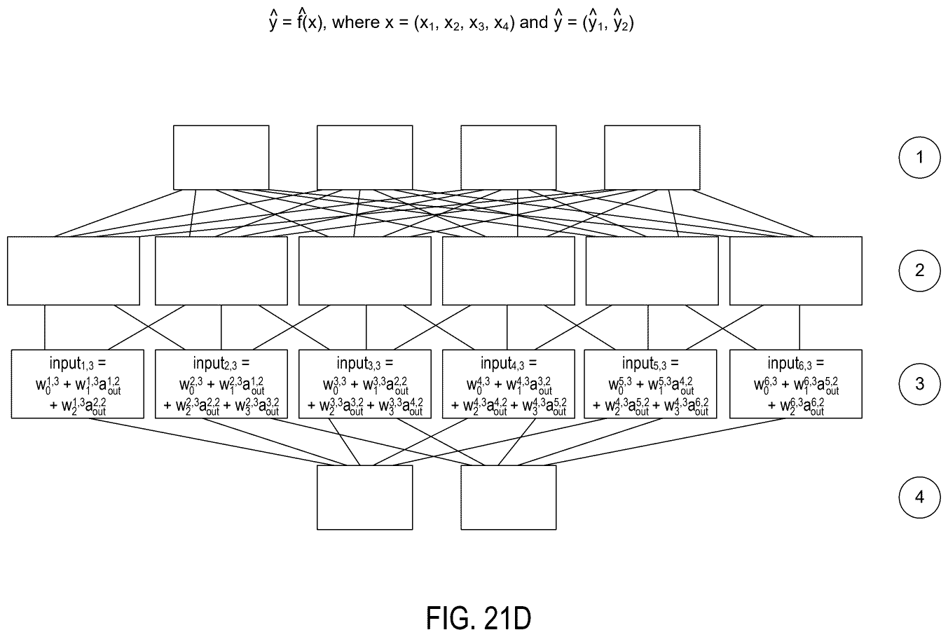

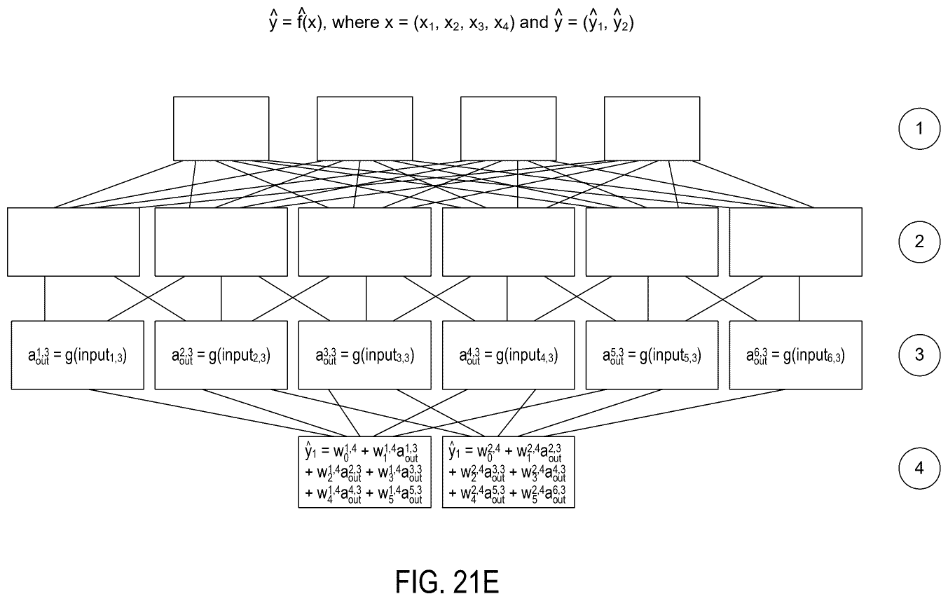

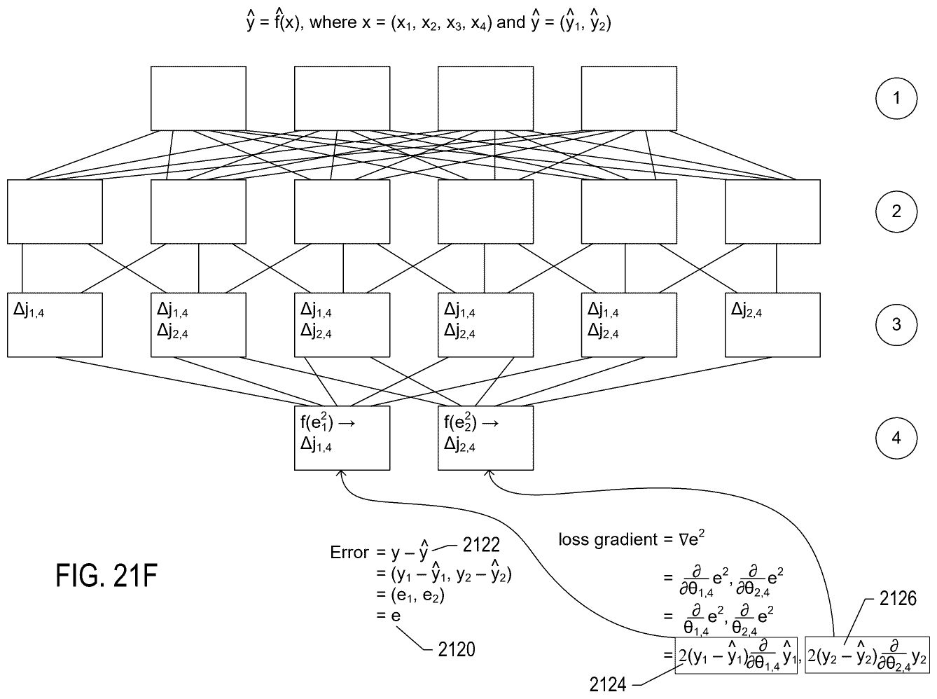

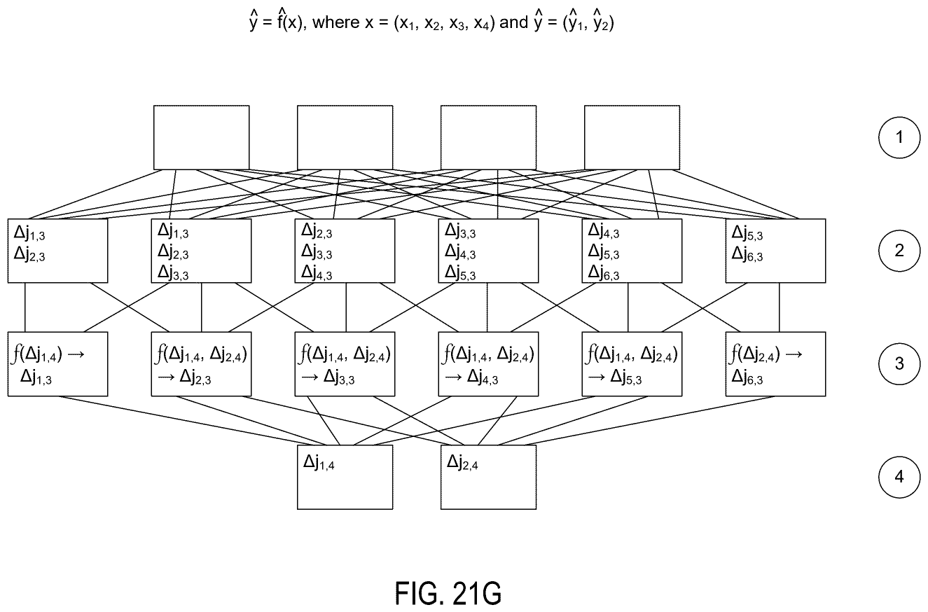

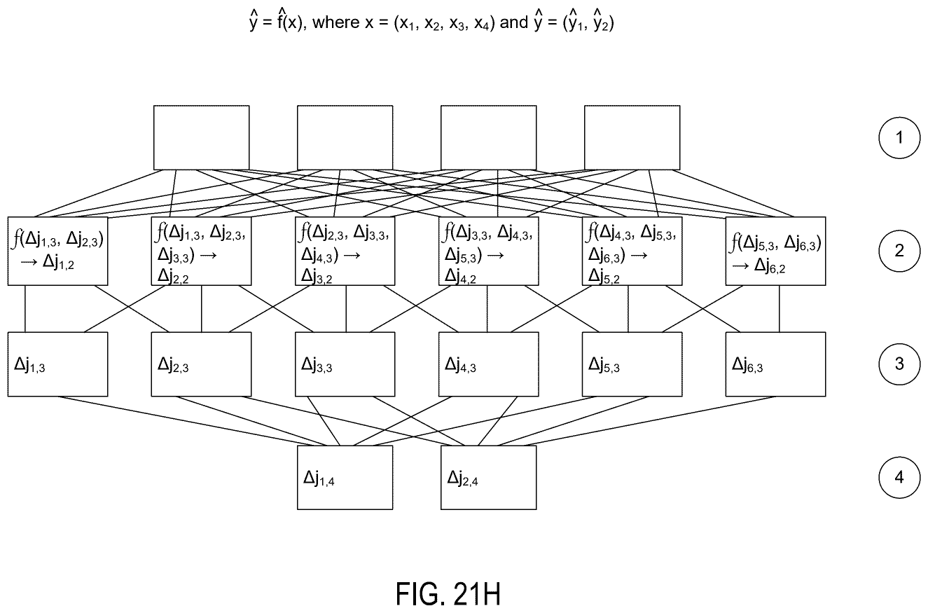

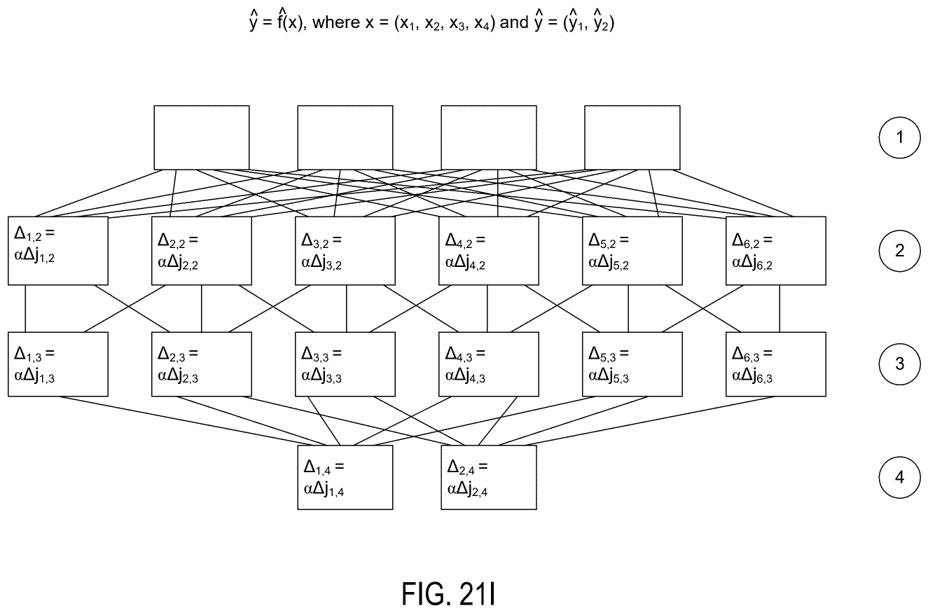

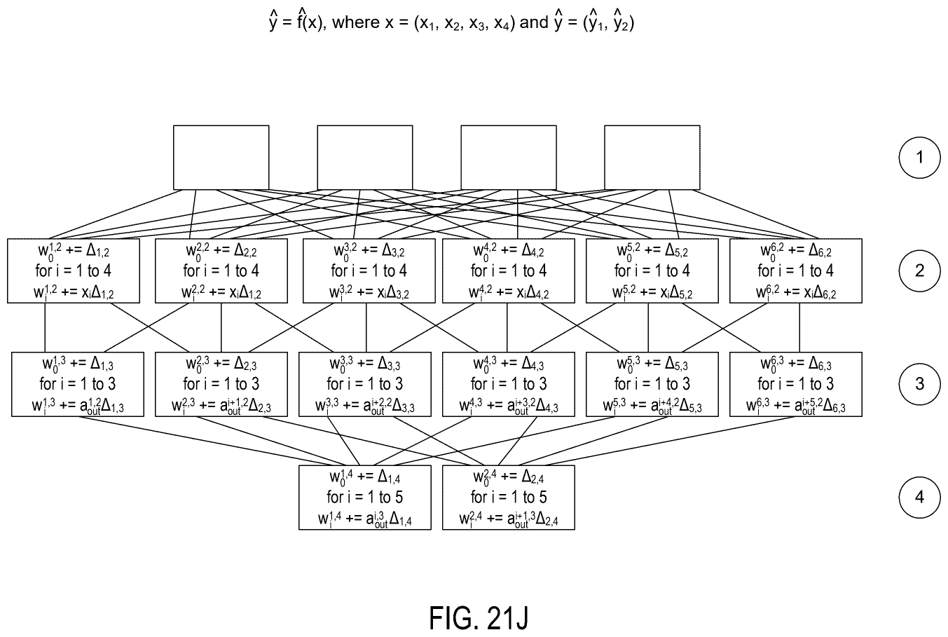

FIGS. 21A-J illustrate operation of a very small, example neural network. The example neural network has four input nodes in a first layer 2102, six nodes in a first hidden layer 2104 six nodes in a second hidden layer 2106, and two output nodes 2108. As shown in FIG. 21A, the four elements of the input vector x 2110 are each input to one of the four input nodes which then output these input values to the nodes of the first-hidden layer to which they are connected. In the example neural network, each input node is connected to all of the nodes in the first hidden layer. As a result, each node in the first hidden layer has received the four input-vector elements, as indicated in FIG. 21A. As shown in FIG. 21B, each of the first-hidden-layer nodes computes a weighted-sum input according to the expression contained in the input components (2036 in FIG. 20) of the first hidden-layer nodes. Note that, although each first-hidden-layer node receives the same four input-vector elements, the weighted-sum input computed by each first-hidden-layer node is generally different from the weighted-sum inputs computed by the other first-hidden-layer nodes, since each first-hidden-layer node generally uses a set of weights unique to the first-hidden-layer node. As shown in FIG. 21C, the activation component (2038 in FIG. 20) of each of the first-hidden-layer nodes next computes an activation and then outputs the computed activation to each of the second-hidden-layer nodes to which the first-hidden-layer node is connected. Thus, for example, the first-hidden-layer node 2112 computes activation

$${a}_{out}^{1 , 2}$$

using the activation function and outputs this activation to second-hidden-layer nodes 2114 and 2116. As shown in FIG. 21D, the input components (2036 in FIG. 20) of the second-hidden-layer nodes compute weighted-sum inputs from the activations received from the first-hidden-layer nodes to which they are connected and then, as shown in FIG. 21E, compute activations from the weighted-sum inputs and output the activations to the output-layer nodes to which they are connected. The output-layer nodes compute weighted sums of the inputs and then output those weighted sums as elements of the output vector.

FIG. 21F illustrates backpropagation of an error computed for an output vector. Backpropagation of a loss in the reverse direction through the neural network results in a change in some or all of the neural-network-node weights and is the mechanism by which a neural network is trained. The error vector e 2120 is computed as the difference between the desired output vector y and the output vector ŷ (2122 in FIG. 21F) produced by the neural network in response to input of the vector x. The output-layer nodes each receive a squared element of the error vector and compute a component of a gradient of the squared length of the error vector with respect to the parameters θ of the neural-network, which are the weights. Thus, in the current example, the squared length of the error vector e is equal to |e|² or

$${e}_{1}^{2} + {e}_{2}^{2} ,$$

and the loss gradient is equal to:

$${∇}_{θ} ( {e}_{1}^{2} + {e}_{2}^{2} ) = \frac{\partial }{\partial θ} {e}_{1}^{2} , \frac{\partial }{\partial θ} {e}_{2}^{2} .$$

Since each output-layer neural-network node represents one dimension of the multi-dimensional output, each output-layer neural-network node receives one term of the squared distance of the error vector and computes the partial differential of that term with respect to the parameters, or weights, of the output-layer neural-network node. Thus, the first output-layer neural-network node receives

$${e}_{1}^{2}$$

and computes

$$\frac{\partial }{\partial {θ}_{1 , 4}} {e}_{1}^{2} ,$$

where the subscript 1,4 indicates parameters for the first node of the fourth, or output, layer. The output-layer neural-network nodes then compute this partial derivative, as indicated by expressions 2124 and 2126 in FIG. 21F. The computations are discussed later. However, to follow the backpropagation diagrammatically, each node of the output layer receives a term of the squared length of the error vector which is input to a function that returns a weight adjustment Δⱼ. As shown in FIG. 21F, the weight adjustment computed by each of the output nodes is back propagated upward to the second-hidden-layer nodes to which the output node is connected. Next, as shown in FIG. 21G, each of the second-hidden-layer nodes computes a weight adjustment Δⱼ from the weight adjustments received from the output-layer nodes and propagates the computed weight adjustments upward in the neural network to the first-hidden-layer nodes to which the second-hidden-layer node is connected. Finally, as shown in FIG. 21H, the first-hidden-layer nodes computes weight adjustments based on the weight adjustments received from the second-hidden-layer nodes. These weight adjustments are not, however, back propagated further upward in the neural network since the input-layer nodes do not compute weighted sums of input activations, instead each receiving only a single element of the input vector x.

In a next logical step, shown in FIG. 21I, the computed weight adjustments are multiplied by a learning constant α to produce final weight adjustments Δ for each node in the neural network. In general, each final weight adjustment is specific and unique for each neural-network node, since each weight adjustment is computed based on a node's weights and the weights of lower-level nodes connected to a node via a path in the neural network. The logical step shown in FIG. 21I is not, in practice, a separate discrete step since the final weight adjustments can be computed immediately following computation of the initial weight adjustment by each node. Similarly, as shown in FIG. 21J, in a final logical step, each node adjusts its weights using the computed final weight adjustment for the node. Again, this final logical step is, in practice, not a discrete separate step since a node can adjust its weights as soon as the final weight adjustment for the node is computed. It should be noted that the weight adjustment made by each node involves both the final weight adjustment computed by the node as well as the inputs received by the node during computation of the output vector ŷ from which the error vector e was computed, as discussed above with reference to FIG. 21F. The weight adjustment carried out by each node shift the weights in each node toward producing an output that, together with the outputs produced by all the other nodes following weight adjustment, results in decreasing the distance between the desired output vector y and the output vector ŷ that would now be produced by the neural network in response to receiving the input vector x. In many neural-network implementations, it is possible to make batched adjustments to the neural-network weights based on multiple output vectors produced from multiple inputs, as discussed further below.

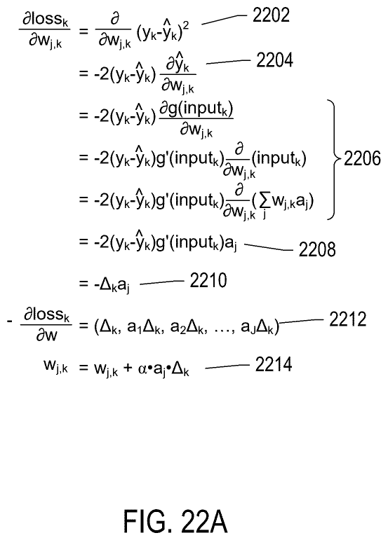

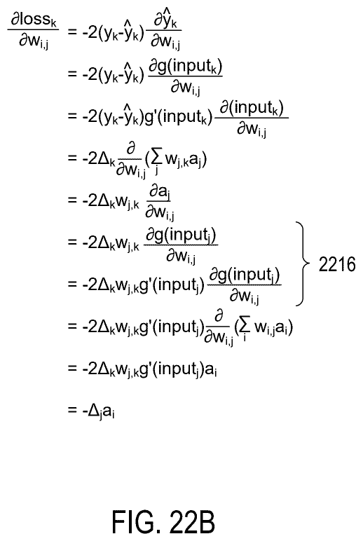

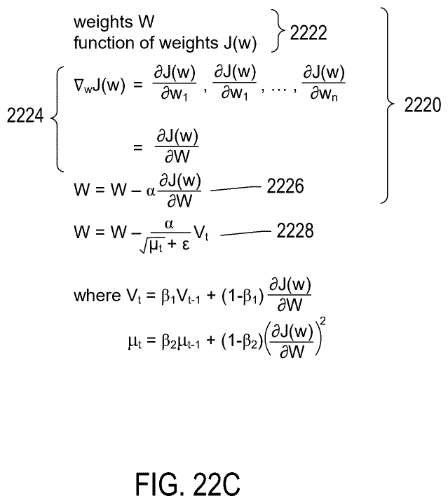

FIGS. 22A-C show details of the computation of weight adjustments made by neural-network nodes during backpropagation of error vectors into neural networks. The expression 2202 in FIG. 22A represents the partial differential of the loss, or k^{th} component of the squared length of the error vector

$${e}_{k}^{2} ,$$

computed by the k^{th} output-layer neural-network node with respect to the J+1 weights applied to the formal 0^{th} input a₀ and inputs a₁-a_{J} received from higher-level nodes. Application of the chain rule for partial differentiation produces expression 2204. Substitution of the activation function for ŷₖ in the second application of the chain rule produces expressions 2206. The partial differential of the sum of weighted activations with respect to the weight for activation j is simply activation j, aⱼ, generating expression 2208. The initial factors in expression 2208 are replaced by −Δₖ to produce a final expression for the partial differential of the k^{th} component of the loss with respect to the j^{th} weight, 2210. The negative gradient of the weight adjustments is used in backpropagation in order to minimize the loss, as indicated by expression 2212. Thus, the j^{th} weight for the k^{th} output-layer neural-network node is adjusted according to expression 2214, where α is a learning-rate constant in the range [0,1].

FIG. 22B illustrates computation of the weight adjustment for the kth component of the error vector in a final-hidden-layer neural-network node. This computation is similar to that discussed above with reference to FIG. 22A, but includes an additional application of the chain rule for partial differentiation in expressions 2216 in order to obtain an expression for the partial differential with respect to a second-hidden-layer-node weight that includes an output-layer-node weight adjustment.

FIG. 22C illustrates one commonly used improvement over the above-described weight-adjustment computations. The above-described weight-adjustment computations are summarized in expressions 2220. There is a set of weights W and a function of the weights J(W), as indicated by expressions 2222. The backpropagation of errors through the neural network is based on the gradient, with respect to the weights, of the function J(W), as indicated by expressions 2224. The weight adjustment is represented by expression 2226, in which a learning constant times the gradient of the function J(W) is subtracted from the weights to generate the new, adjusted weights. In the improvement illustrated in FIG. 22C, expression 2226 is modified to produce expression 2228 for the weight adjustment. In the improved weight adjustment, the learning constant α is divided by the sum of a weighted average of adjustments and a very small additional term ε and the gradient is replaced by the factor Vₜ, where t represents time or, equivalently, the current weight adjustment in a series of weight adjustments. The factor Vₜ is a combination of the factor for the preceding time point or weight adjustment Vₜ₋₁ and the gradient computed for the current time point or weight adjustment. This factor is intended to add momentum to the gradient descent in order to avoid premature completion of the gradient-descent process at a local minimum. Division of the learning constant α by the weighted average of adjustments adjusts the learning rate over the course of the gradient descent so that the gradient descent converges in a reasonable period of time.

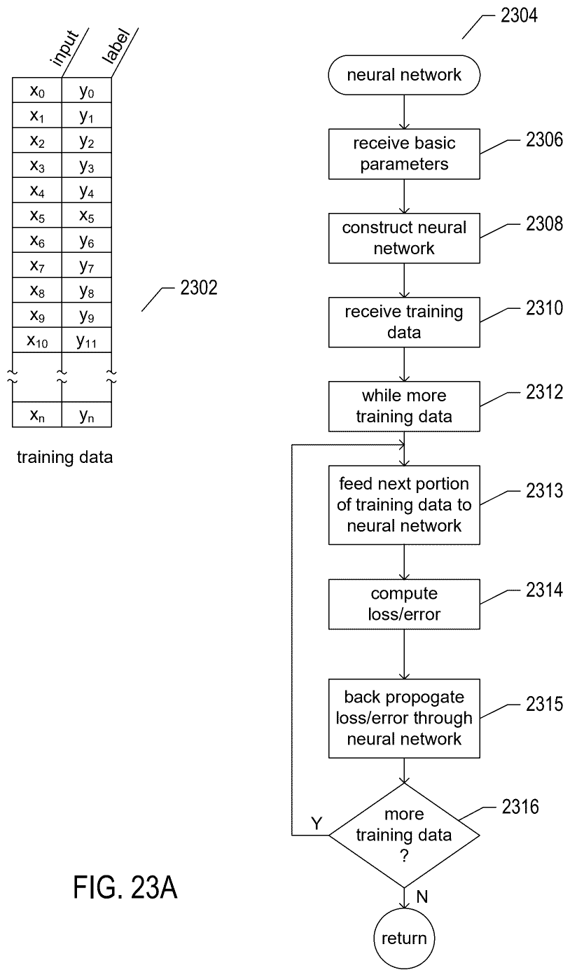

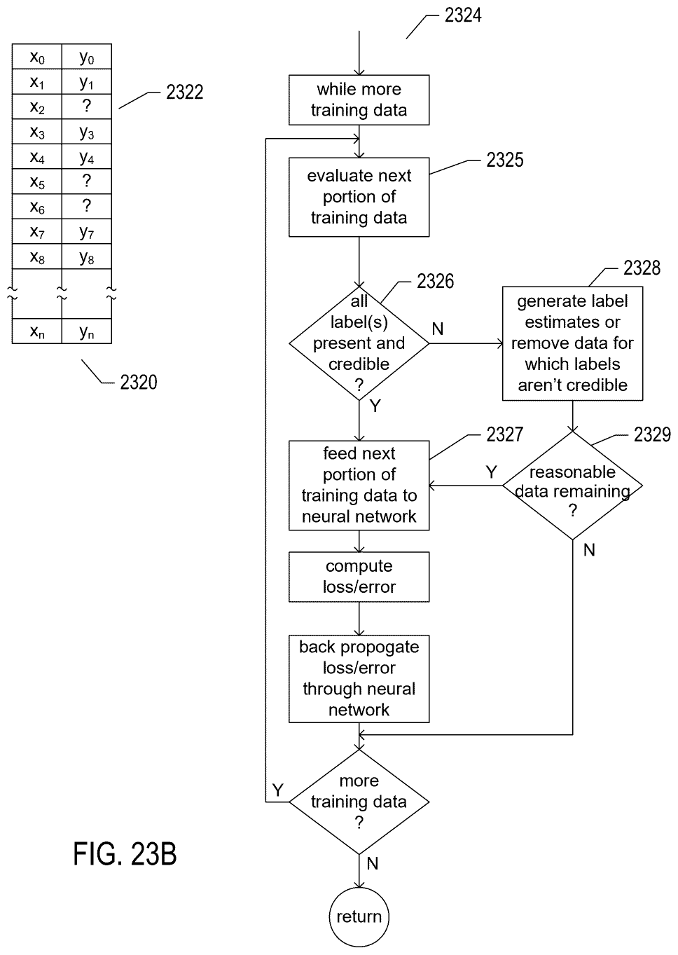

FIGS. 23A-B illustrate neural-network training. FIG. 23A illustrates the construction and training of a neural network using a complete and accurate training dataset. The training dataset is shown as a table of input-vector/label pairs 2302, in which each row represents an input-vector/label pair. The control-flow diagram 2304 illustrates construction and training of a neural network using the training dataset. In step 2306, basic parameters for the neural network are received, such as the number of layers, number of nodes in each layer, node interconnections, and activation functions. In step 2308, the specified neural network is constructed. This involves building representations of the nodes, node connections, activation functions, and other components of the neural network in one or more electronic memories and may involve, in certain cases, various types of code generation, resource allocation and scheduling, and other operations to produce a fully configured neural network that can receive input data and generate corresponding outputs. In many cases, for example, the neural network may be distributed among multiple computer systems and may employ dedicated communications and shared memory for propagation of activations and total error or loss between nodes. It should again be emphasized that a neural network is a physical system comprising one or more computer systems, communications subsystems, and often multiple instances of computer-instruction-implemented control components.

In step 2310, training data represented by table 2302 is received. Then, in the while-loop of steps 2312-2316, portions of the training data are iteratively input to the neural network, in step 2313, the loss or error is computed, in step 2314, and the computed loss or error is back-propagated through the neural network step 2315 to adjust the weights. The control-flow diagram refers to portions of the training data rather than individual input-vector/label pairs because, in certain cases, groups of input-vector/label pairs are processed together to generate a cumulative error that is back-propagated through the neural network. A portion may, of course, include only a single input-vector/label pair.

FIG. 23B illustrates one method of training a neural network using an incomplete training dataset. Table 2320 represents the incomplete training dataset. For certain of the input-vector/label pairs, the label is represented by a “?” symbol, such as in the input-vector/label pair 2322. The “?” symbol indicates that the correct value for the label is unavailable. This type of incomplete data set may arise from a variety of different factors, including inaccurate labeling by human annotators, various types of data loss incurred during collection, storage, and processing of training datasets, and other such factors. The control-flow diagram 2324 illustrates alterations in the while-loop of steps 2312-2316 in FIG. 23A that might be employed to train the neural network using the incomplete training dataset. In step 2325, a next portion of the training dataset is evaluated to determine the status of the labels in the next portion of the training data. When all of the labels are present and credible, as determined in step 2326, the next portion of the training dataset is input to the neural network, in step 2327, as in FIG. 23A. However, when certain labels are missing or lack credibility, as determined in step 2326, the input-vector/label pairs that include those labels are removed or altered to include better estimates of the label values, in step 2328. When there is reasonable training data remaining in the training-data portion following step 2328, as determined in step 2329, the remaining reasonable data is input to the neural network in step 2327. The remaining steps in the while-loop are equivalent to those in the control-flow diagram shown in FIG. 23A. Thus, in this approach, either suspect data is removed, or better labels are estimated, based on various criteria, for substitution for the suspect labels.

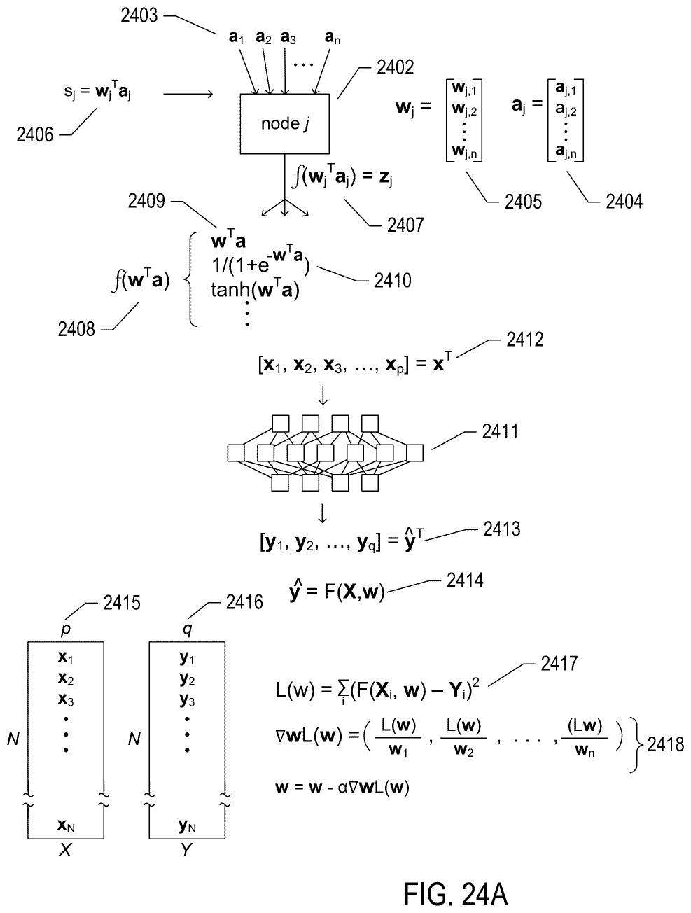

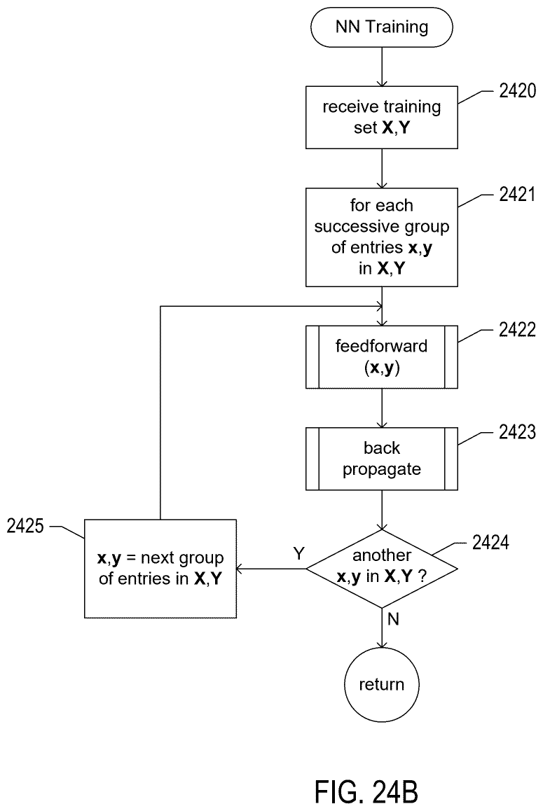

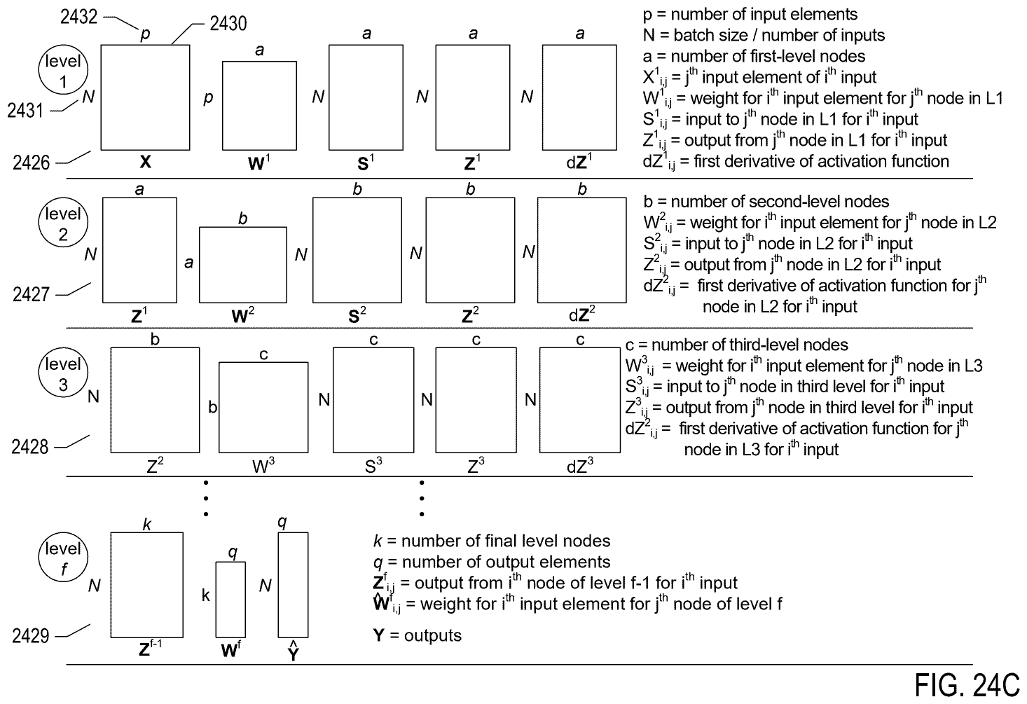

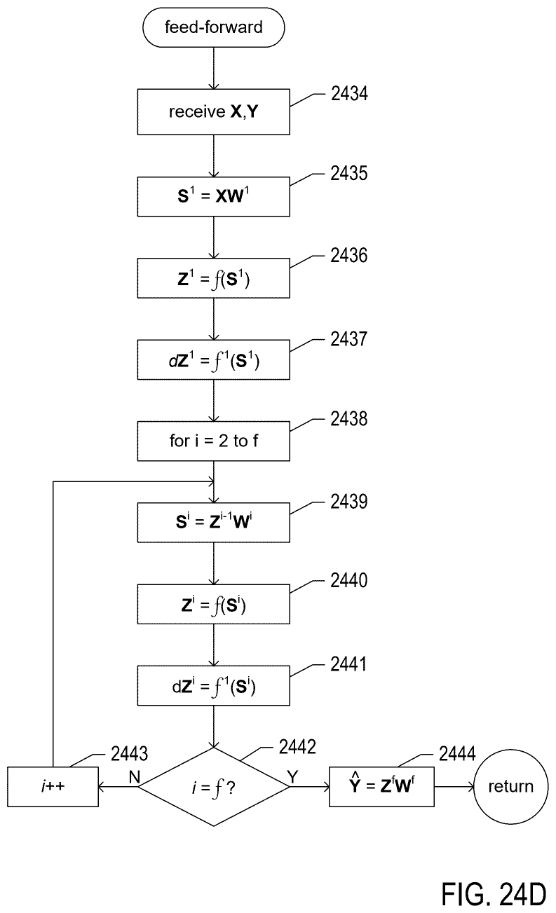

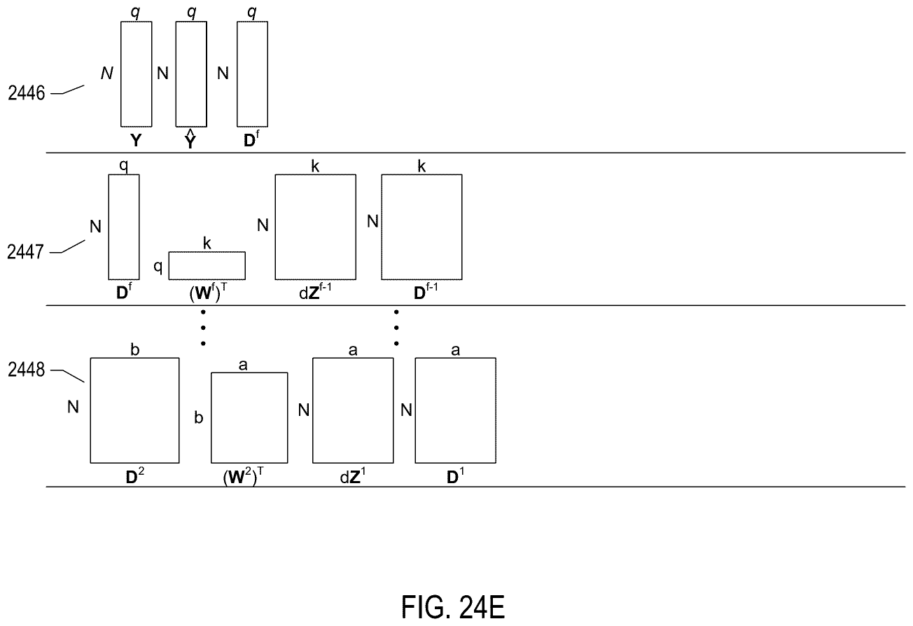

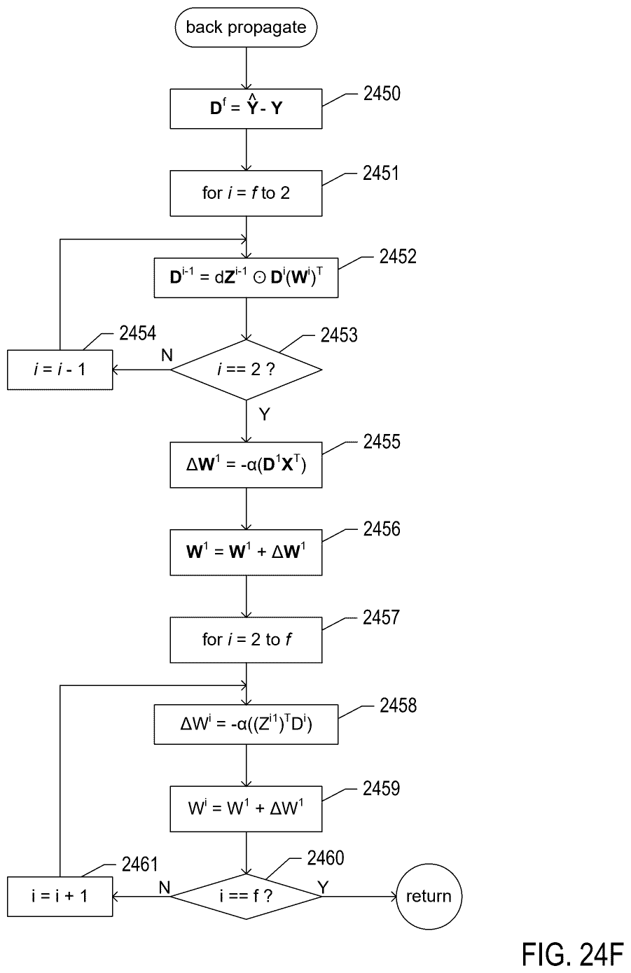

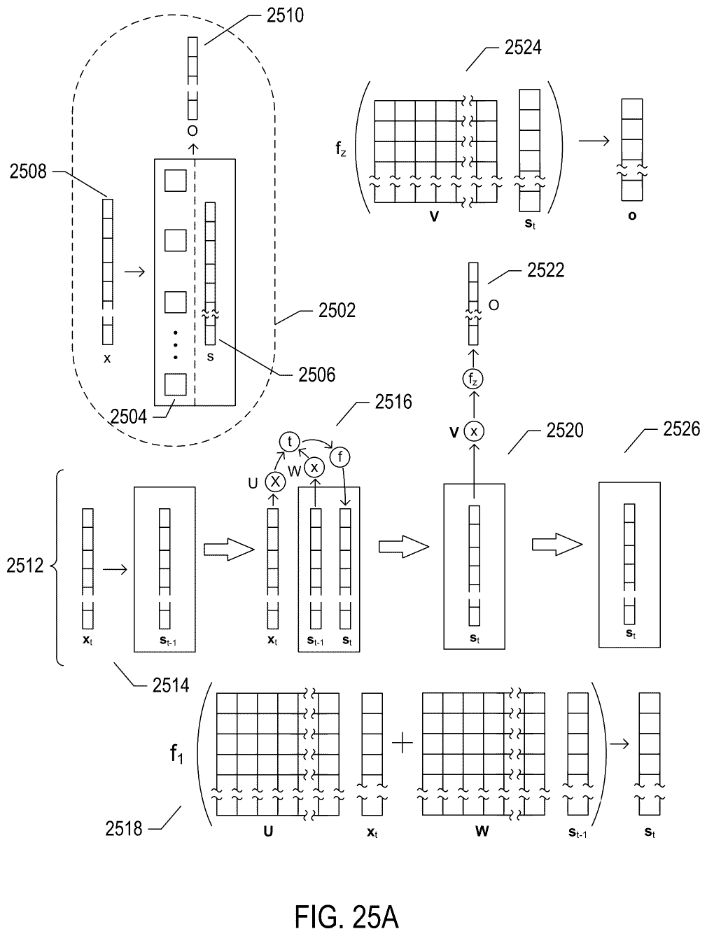

FIGS. 24A-F illustrate a matrix-operation-based batch method for neural-network training. This method processes batches of training data and losses to efficiently train a neural network. FIG. 24A illustrates the neural network and associated terminology. As discussed above, each node in the neural network, such as node j 2402, receives one or more inputs a 2403, expressed as a vector aⱼ 2404, that are multiplied by corresponding weights, expressed as a vector wⱼ 2405, and added together to produce an input signal sⱼ using a vector dot-product operation 2406. An activation function f within the node receives the input signal sⱼ and generates an output signal zⱼ 2407 that is output to all child nodes of node j. Expression 2408 provides an example of various types of activation functions that may be used in the neural network. These include a linear activation function 2409 and a sigmoidal activation function 2410. As discussed above, the neural network 2411 receives a vector of p input values 2412 and outputs a vector of q output values 2413. In other words, the neural network can be thought of as a function F 2414 that receives a vector of input values x^{T} and uses a current set of weights w within the nodes of the neural network to produce a vector of output values ŷ^{T}. The neural network is trained using a training data set comprising a matrix X 2415 of input values, each of N rows in the matrix corresponding to an input vector x^{T}, and a matrix Y 2416 of desired output values, or labels, each of N rows in the matrix corresponding to a desired output-value vector y^{T}. A least-squares loss function is used in training 2417 with the weights updated using a gradient vector generated from the loss function, as indicated in expressions 2418, where a is a constant that corresponds to a learning rate.

FIG. 24B provides a control-flow diagram illustrating the method of neural-network training. In step 2420, the routine “NNTraining” receives the training set comprising matrices X and Y. Then, in the for-loop of steps 2421-2425, the routine “NNTraining” processes successive groups or batches of entries x and y selected from the training set. In step 2422, the routine “NNTraining” calls a routine “feedforward” to process the current batch of entries to generate outputs and, in step 2423, calls a routine “back propagated” to propagate errors back through the neural network in order to adjust the weights associated with each node.

FIG. 24C illustrates various matrices used in the routine “feedforward.” FIG. 24C is divided horizontally into four regions 2426-2429. Region 2426 approximately corresponds to the input level, regions 2427-2428 approximately correspond to hidden-node levels, and region 2429 approximately corresponds to the final output level. The various matrices are represented, in FIG. 24C, as rectangles, such as rectangle 2430 representing the input matrix X. The row and column dimensions of each matrix are indicated, such as the row dimension N 2431 and the column dimension p 2432 for input matrix X 2430. In the right-hand portion of each region in FIG. 24C, descriptions of the matrix-dimension values and matrix elements are provided. In short, the matrices W^{x} represent the weights associated with the nodes at level x, the matrices S^{x} represent the input signals associated with the nodes at level x, the matrices Z^{x} represent the outputs from the nodes at level x, and the matrices dZ^{x} represent the first derivative of the activation function for the nodes at level x evaluated for the input signals.

FIG. 24D provides a control-flow diagram for the routine “feedforward,” called in step 2422 of FIG. 24B. In step 2434, the routine “feedforward” receives a set of training data x and y selected from the training-data matrices X and Y. In step 2435, the routine “feedforward” computes the input signals S¹ for the first layer of nodes by matrix multiplication of matrices x and W¹, where matrix W¹ contains the weights associated with the first-layer nodes. In step 2436, the routine “feedforward” computes the output signals Z¹ for the first-layer nodes by applying a vector-based activation function f to the input signals S¹. In step 2437, the routine “feedforward” computes the values of the derivatives of the activation function f′, dZ¹. Then, in the for-loop of steps 2438-2443, the routine “feedforward” computes the input signals Sⁱ, the output signals Zⁱ, and the derivatives of the activation function dZⁱ for the nodes of the remaining levels of the neural network. Following completion of the for-loop of steps 2438-2443, the routine “feedforward” computes the output values ŷ^{T} for the received set of training data.

FIG. 24E illustrates various matrices used in the routine “back propagate.” FIG. 24E uses similar illustration conventions as used in FIG. 24C, and is also divided horizontally into horizontal regions 2446-2448. Region 2446 approximately corresponds to the output level, region 2447 approximately corresponds to hidden-node levels, and region 2448 approximately corresponds to the first node level. The only new type of matrix shown in FIG. 24E are the matrices D^{x} for node levels x. These matrices contain the error signals that are used to adjust the weights of the nodes.

FIG. 24F provides a control-flow diagram for the routine “back propagate.” In step 2450, the routine “back propagate” computes the first error-signal matrix D^{f} as the difference between the values ŷ output during a previous execution of the routine “feedforward” and the desired output values from the training set y. Then, in a for-loop of steps 2451-2454, the routine “back propagate” computes the remaining error-signal matrices for each of the node levels up to the first node level as the Shur product of the dZ matrix and the product of the transpose of the W matrix and the error-signal matrix for the next lower node level. In step 2455, the routine “back propagate” computes weight adjustments ΔW for the first-level nodes as the negative of the constant α times the product of the transpose of the input-value matrix and the error-signal matrix. In step 2456, the first-node-level weights are adjusted by adding the current W matrix and the weight-adjustments matrix ΔW. Then, in the for-loop of steps 2457-2461, the weights of the remaining node levels are similarly adjusted.

Thus, as shown in FIGS. 24A-F, neural-network training can be conducted as a series of simple matrix operations, including matrix multiplications, matrix transpose operations, matrix addition, and the Shur product. Interestingly, there are no matrix inversions or other complex matrix operations needed for neural-network training.

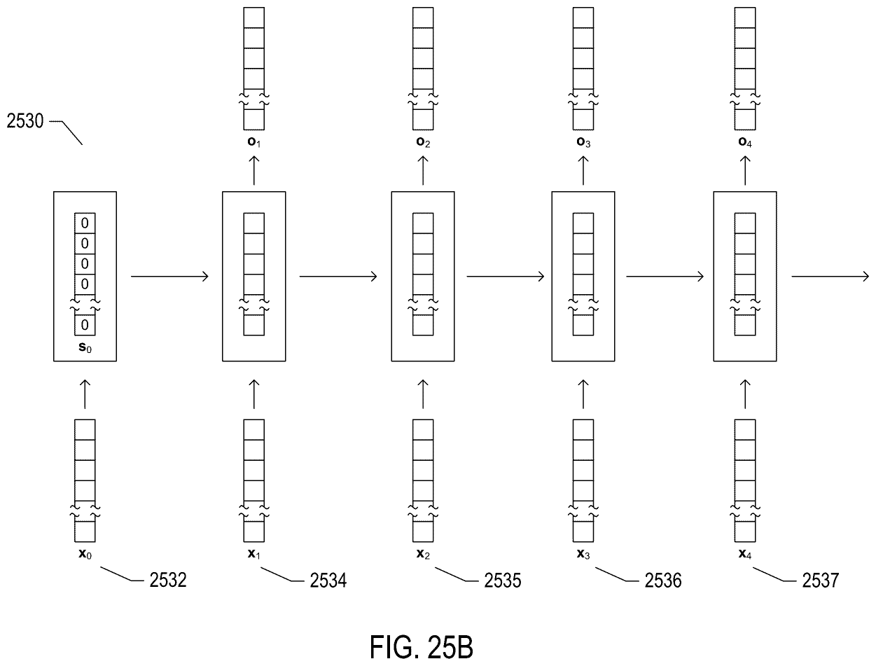

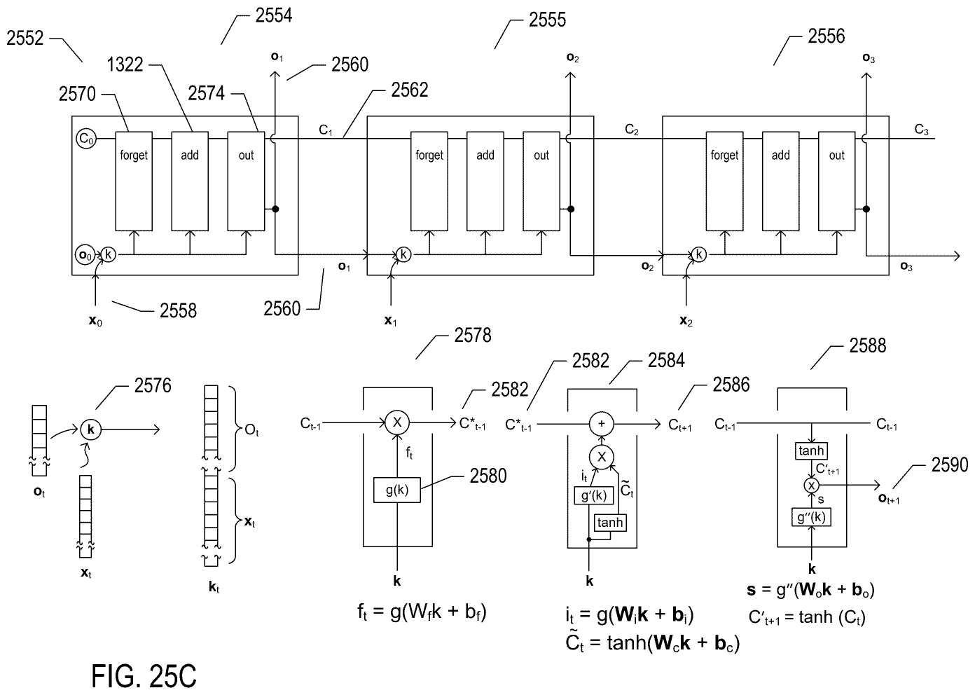

A second type of neural network, referred to as a “recurrent neural network,” is employed to generate sequences of output vectors from sequences of input vectors. These types of neural networks are often used for natural-language applications in which a sequence of words forming a sentence are sequentially processed to produce a translation of the sentence, as one example. FIGS. 25A-B illustrate various aspects of recurrent neural networks. Inset 2502 in FIG. 25A shows a representation of a set of nodes within a recurrent neural network. The set of nodes includes nodes that are implemented similarly to those discussed above with respect to the feed-forward neural network 2504, but additionally include an internal state 2506. In other words, the nodes of a recurrent neural network include a memory component. The set of recurrent-neural-network nodes, at a particular time point in a sequence of time points, receives an input vector x 2508 and produces an output vector 2510. The process of receiving an input vector and producing an output vector is shown in the horizontal set of recurrent-neural-network-nodes diagrams interleaved with large arrows 2512 in FIG. 25A. In a first step 2514, the input vector x at time t is input to the set of recurrent-neural-network nodes which include an internal state generated at time t−1. In a second step 2516, the input vector is multiplied by a set of weights U and the current state vector is multiplied by a set of weights W to produce two vector products which are added together to generate the state vector for time t. This operation is illustrated as a vector function f₁ 2518 in the lower portion of FIG. 25A. In a next step 2520, the current state vector is multiplied by a set of weights V to produce the output vector for time t 2522, a process illustrated as a vector function f₂ 2524 in FIG. 25A. Finally, the recurrent-neural-network nodes are ready for input of a next input vector at time t+1, in step 2526.

FIG. 25B illustrates processing by the set of recurrent-neural-network nodes of a series of input vectors to produce a series of output vectors. At a first time t₀ 2530, a first input vector x₀ 2532 is input to the set of recurrent-neural-network nodes. At each successive time point 2534-2537, a next input vector is input to the set of recurrent-neural-network nodes and an output vector is generated by the set of recurrent-neural-network nodes. In many cases, only a subset of the output vectors is used. Back propagation of the error or loss during training of a recurrent neural network is similar to back propagation for a feed-forward neural network, except that the total error or loss needs to be back-propagated through time in addition to through the nodes. of the recurrent neural network. This can be accomplished by unrolling the recurrent neural network to generate a sequence of component neural networks and by then backpropagating the error or loss through this sequence of component neural networks from the most recent time to the most distant time period.

Finally, for completeness, FIG. 25C illustrates a type of recurrent-neural-network node referred to as a long-short-term-memory (“LSTM”) node. In FIG. 25C, a LSTM node 2552 is shown at three successive points in time 2554-2556. State vectors and output vectors appear to be passed between different nodes, but these horizontal connections instead illustrate the fact that the output vector and state vector are stored within the LSTM node at one point in time for use at the next point in time. At each time point, the LSTM node receives an input vector 2558 and outputs an output vector 2560. In addition, the LSTM node outputs a current state 2562 forward in time. The LSTM node includes a forget module 2570, an add module 2572, and an out module 2574. Operations of these modules are shown in the lower portion of FIG. 25C. First, the output vector produced at the previous time point and the input vector received at a current time point are concatenated to produce a vector k 2576. The forget module 2578 computes a set of multipliers 2580 that are used to element-by-element multiply the state from time t−1 in order to produce an altered state 2582. This allows the forget module to delete or diminish certain elements of the state vector. The add module 2134 employs an activation function to generate a new state 2586 from the altered state 2582. Finally, the out module 2588 applies an activation function to generate an output vector 2140 based on the new state and the vector k. An LSTM node, unlike the recurrent-neural-network node illustrated in FIG. 25A, can selectively alter the internal state to reinforce certain components of the state and deemphasize or forget other components of the state in a manner reminiscent of human short-term memory. As one example, when processing a paragraph of text, the LSTM node may reinforce certain components of the state vector in response to receiving new input related to previous input but may diminish components of the state vector when the new input is unrelated to the previous input, which allows the LSTM to adjust its context to emphasize inputs close in time and to slowly diminish the effects of inputs that are not reinforced by subsequent inputs. Here again, back propagation of a total error or loss is employed to adjust the various weights used by the LSTM, but the back propagation is significantly more complicated than that for the simpler recurrent neural-network nodes discussed with reference to FIG. 25A.

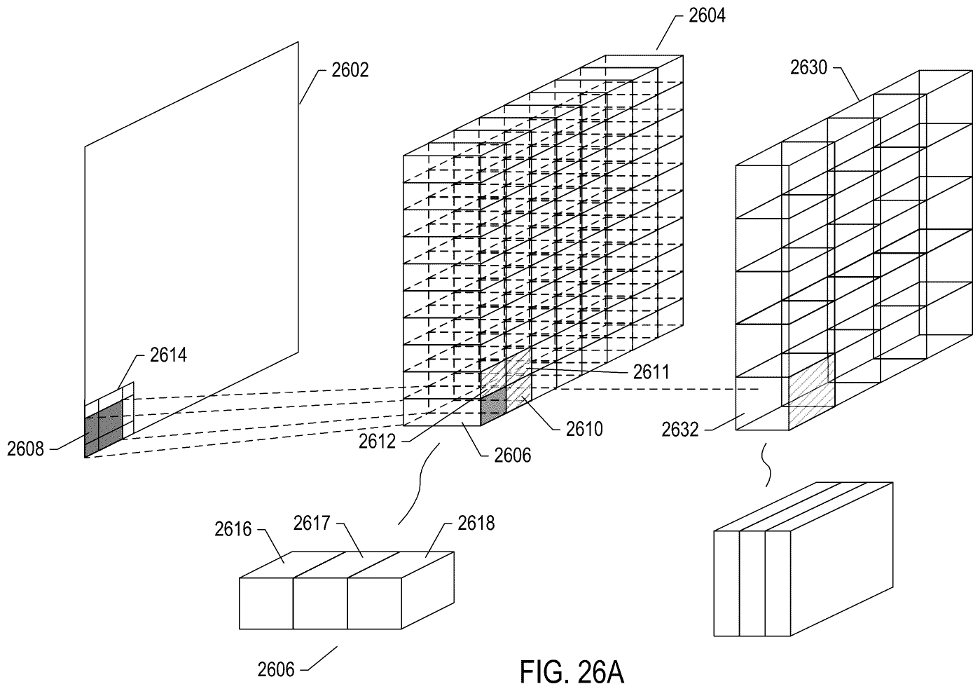

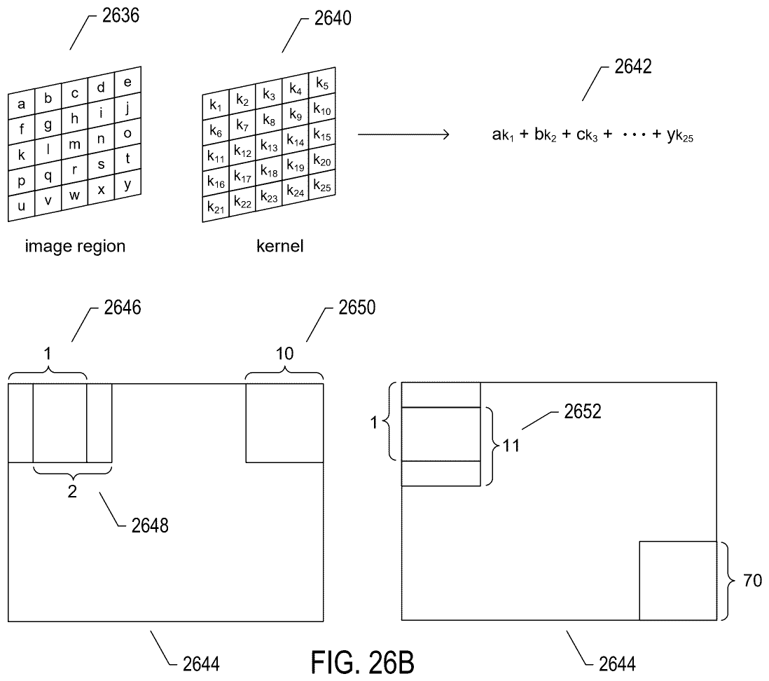

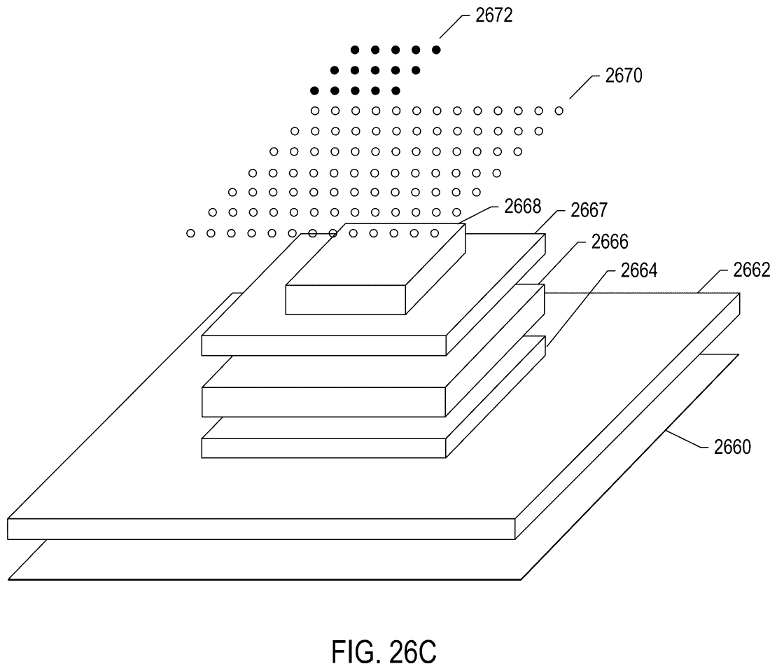

FIGS. 26A-C illustrate a convolutional neural network. Convolutional neural networks are currently used for image processing, voice recognition, and many other types of machine-learning tasks for which traditional neural networks are impractical. In FIG. 26A, a digitally encoded screen-capture image 2602 represents the input data for a convolutional neural network. A first level of convolutional-neural-network nodes 2604 each process a small subregion of the image. The subregions processed by adjacent nodes overlap. For example, the corner node 2606 processes the shaded subregion 2608 of the input image. The set of four nodes. 2606 and 2610-2612 together process a larger subregion 2614 of the input image. Each node may include multiple subnodes. For example, as shown in FIG. 26A, node 2606 includes 3 subnodes 2616-2618. The subnodes within a node all process the same region of the input image, but each subnode may differently process that region to produce different output values. Each type of subnode in each node in the initial layer of nodes 2604 uses a common kernel or filter for subregion processing, as discussed further below. The values in the kernel or filter are the parameters, or weights, that are adjusted during training. However, since all the nodes in the initial layer use the same three subnode kernels or filters, the initial node layer is associated with only a comparatively small number of adjustable parameters. Furthermore, the processing associated with each kernel or filter is more or less translationally invariant, so that a particular feature recognized by a particular type of subnode kernel is recognized anywhere within the input image that the feature occurs. This type of organization mimics the organization of biological image-processing systems. A second layer of nodes 2630 may operate as aggregators, each producing an output value that represents the output of some function of the corresponding output values of multiple nodes in the first node layer 2604. For example, second-a layer node 2632 receives, as input, the output from four first-layer nodes 2606 and 2610-2612 and produces an aggregate output. As with the first-level nodes, the second-level nodes also contain subnodes, with each second-level subnode producing an aggregate output value from outputs of multiple corresponding first-level subnodes.

FIG. 26B illustrates the kernel-based or filter-based processing carried out by a convolutional neural network node. A small subregion of the input image 2636 is shown aligned with a kernel or filter 2640 of a subnode of a first-layer node that processes the image subregion. Each pixel or cell in the image subregion 2636 is associated with a pixel value. Each corresponding cell in the kernel is associated with a kernel value, or weight. The processing operation essentially amounts to computation of a dot product 2642 of the image subregion and the kernel, when both are viewed as vectors. As discussed with reference to FIG. 26A, the nodes of the first level process different, overlapping subregions of the input image, with these overlapping subregions essentially tiling the input image. For example, given an input image represented by rectangles 2644, a first node processes a first subregion 2646, a second node may process the overlapping, right-shifted subregion 2648, and successive nodes may process successively right-shifted subregions in the image up through a tenth subregion 2650. Then, a next down-shifted set of subregions, beginning with an eleventh subregion 2652, may be processed by a next row of nodes.

FIG. 26C illustrates the many possible layers within the convolutional neural network. The convolutional neural network may include an initial set of input nodes 2660, a first convolutional node layer 2662, such as the first layer of nodes 2604 shown in FIG. 26A, and aggregation layer 2664, in which each node processes the outputs for multiple nodes in the convolutional node layer 2662, and additional types of layers 2666-2668 that include additional convolutional, aggregation, and other types of layers. Eventually, the subnodes in a final intermediate layer 2668 are expanded into a node layer 2670 that forms the basis of a traditional, fully connected neural-network portion with multiple node levels of decreasing size that terminate with an output-node level 2672.

The present invention has been described in terms of particular embodiments, but it is not intended that the invention be limited to these embodiments. Modifications within the spirit of the invention will be apparent to those skilled in the art. For example, any of many different implementations of the currently disclosed methods and systems can be obtained by varying various design and implementation parameters, including modular organization, control structures, data structures, hardware, operating system, and virtualization layers, and other such design and implementation parameters. Many alternative implementations are possible. For example, different types of neural networks can be used for implementing generic efficacy-estimation functions and severity functions. Currently disclosed methods and systems can be used to provide treatments for a wide variety of different types of medical conditions using a wide variety of different types of treatments, treatment devices and treatment facilities. As another example, while the above discussion describes treatment plans as being control-variable vectors, observation and monitoring data as observation-data vectors, and patient-specific/treatment-specific patient information as pti vectors, these data collections need not be formatted as vectors in implementations of the currently disclosed methods and systems, but can instead be considered as ordered sets or collections. As another example, rather than using neural networks to implement generic efficacy-estimation functions, patient-specific efficacy-estimation functions, and severity functions, other types of implementations can instead be used, including transformers and large-language models, rule-based systems, and decision-tree based systems. Many implementations may implement the functions using combinations of different types of data-generated inferential systems.

1. A treatment method that provides a personalized medical treatment or therapy to a new patient, the treatment method comprising:
receiving the new patient;
acquiring patient information from the new patient;
using the acquired patient information to determine a treatment type and to construct a patient-treatment-information instance;
using the determined treatment type to determine a first treatment plan for the new patient by optimizing a set of control variables representing the treatment plan using a generic efficacy-estimation function that receives, as inputs, the set of control variables and the patient-treatment-information instance;
carrying out a number of experiments by
for each experiment,
carrying out a next experimental treatment using a next treatment plan, wherein the next treatment plan is the first treatment plan for the first experiment and a most recently generated next treatment plan for subsequent experiments,
determining a treatment efficacy,
storing the determined treatment efficacy and the next treatment plan,
generating a new patient-specific efficacy-estimation function as a deformation of the generic efficacy-estimation function, and
using the new patient-specific efficacy-estimation function to optimize a set of control variables representing a treatment plan to generate a new next treatment plan; and
selecting a treatment plan from among the next treatment plan generated following a final treatment experiment and a stored treatment plan; and
applying a treatment of the determined treatment type and specified by the selected treatment plan to the new patient.

2. The treatment method of claim 1 wherein both the generic efficacy-estimation function and each patient-specific efficacy-estimation function receive, as inputs, a set of control variables and a patient-treatment-information instance, and output an estimate of the treatment efficacy that would obtain were the patient represented by the patient-treatment-information instance treated according to the control variables.

3. The treatment method of claim 2
wherein the generic efficacy-estimation function is implemented as a neural network, by another type of trainable computational entity, or by a combination of trainable computational entities; and
wherein the generic efficacy-estimation function is trained to produce an estimate of the treatment efficacy that would obtain were the patient represented by the patient-treatment-information instance treated according to the control variables.

4. The treatment method of claim 3 wherein the generic efficacy-estimation function is trained using treatment information collected from many different patients over time periods of days, weeks, months, or years.

5. The treatment method of claim 4 wherein a patient-specific efficacy-estimation function comprises:
the generic efficacy-estimation function;
a control-variable transform; and
an efficacy-estimation modifier.

6. The treatment method of claim 5 wherein the patient-specific efficacy-estimation function:
receives, as inputs, a set of control variables and a patient-treatment-information instance;
uses the control-variable transform to transform the received set of control variables;
inputs the transformed set of control variables to the generic efficacy-estimation function;
receives an efficacy estimate output by the generic efficacy-estimation function;
uses the efficacy-estimation modifier to modify the efficacy estimate output by the generic efficacy-estimation function; and
returns the modified efficacy estimate as output from the patient-specific efficacy-estimation function.

7. The treatment method of claim 6 wherein the efficacy-estimation modifier adds a constant value to the efficacy estimate output by the generic efficacy-estimation function.

8. The treatment method of claim 7 wherein a patient-specific efficacy-estimation function is generated as a deformation of the generic efficacy-estimation function using a constrained optimization process that optimizes the control-variable transform and the efficacy-estimation modifier to align the output of the patient-specific efficacy-estimation function with the stored treatment efficacy or efficacies and treatment plan or plans.

9. The treatment method of claim 1 wherein selecting a treatment plan from among the next treatment plan generated following a final treatment experiment and a stored treatment plan further comprises:
selecting the next treatment plan generated following the final treatment experiment when an aggressive approach is indicated; and
selecting a most effective stored treatment plan from among the stored treatment plans when a conservative approach is indicated.

10. A system that implements the method of claim 1, the system comprising:
one or more of local computer systems and remote computer systems that execute an application the implements the method of claim 1; and
one or more databases that store treatment data and patient data.

11. A treatment method that provides a personalized medical treatment or therapy to a returning patient, the treatment method comprising:
receiving the returning patient;
acquiring patient information from the returning patient;
using the acquired patient information to access stored patient information for the returning patient;
determining a treatment type and constructing a patient-treatment-information instance using the acquired patient information and stored patient information;
using the determined treatment type to determine a first treatment plan for the returning patient by optimizing a set of control variables representing the first treatment plan using one of a generic efficacy-estimation function or a patient-specific efficacy-estimation function previously generated for the returning patient that receives, as inputs, a set of control variables and the patient-treatment-information instance;
carrying out a number of experiments by
for each experiment,
carrying out a next experimental treatment using a next treatment plan, wherein the next treatment plan is the first treatment plan for the first experiment and a most recently generated next treatment plan for subsequent experiments,
determining a treatment efficacy,
storing the determined treatment efficacy and the next treatment plan,
generating a new patient-specific efficacy-estimation function as a deformation of the generic efficacy-estimation function, and
using the new patient-specific efficacy-estimation function to optimize a set of control variables representing a treatment plan to generate a new next treatment plan; and
selecting a treatment plan from among the next treatment plan generated following a final treatment experiment and a stored treatment plan; and
applying a treatment of the determined treatment type and specified by the selected treatment plan to the returning patient.

12. The treatment method of claim 11 wherein both the generic efficacy-estimation function and each patient-specific efficacy-estimation function receive, as inputs, a set of control variables and a patient-treatment-information instance, and output an estimate of the treatment efficacy that would obtain were the patient represented by the patient-treatment-information instance treated according to the control variables.

13. The treatment method of claim 12
wherein the generic efficacy-estimation function is implemented as a neural network, by another type of trainable computational entity, or by a combination of trainable computational entities; and
wherein the generic efficacy-estimation function is trained to produce an estimate of the treatment efficacy that would obtain were the patient represented by the patient-treatment-information instance treated according to the control variables.

14. The treatment method of claim 13 wherein the generic efficacy-estimation function is trained using treatment information collected from many different patients over time periods of days, weeks, months, or years.

15. The treatment method of claim 14 wherein a patient-specific efficacy-estimation function comprises:
the generic efficacy-estimation function;
a control-variable transform; and
an efficacy-estimation modifier.

16. The treatment method of claim 15 wherein the patient-specific efficacy-estimation function:
receives, as inputs, a set of control variables and a patient-treatment-information instance;
uses the control-variable transform to transform the received set of control variables;
inputs the transformed set of control variables to the generic efficacy-estimation function;
receives an efficacy estimate output by the generic efficacy-estimation function;
uses the efficacy-estimation modifier to modify the efficacy estimate output by the generic efficacy-estimation function; and
returns the modified efficacy estimate as output from the patient-specific efficacy-estimation function.

17. The treatment method of claim 16 wherein the efficacy-estimation modifier adds a constant value to the efficacy estimate output by the generic efficacy-estimation function.

18. The treatment method of claim 17 wherein a patient-specific efficacy-estimation function is generated as a deformation of the generic efficacy-estimation function using a constrained optimization process that optimizes the control-variable transform and the efficacy-estimation modifier to align the output of the patient-specific efficacy-estimation function with the stored treatment efficacy or efficacies and treatment plan or plans.

19. The treatment method of claim 11 wherein selecting a treatment plan from among the next treatment plan generated following a final treatment experiment and a stored treatment plan further comprises:
selecting the next treatment plan generated following the final treatment experiment when an aggressive approach is indicated; and
selecting a most effective stored treatment plan from among the stored treatment plans when a conservative approach is indicated.

20. A system that implements the method of claim 1, the system comprising:
one or more of local computer systems and remote computer systems that execute an application the implements the method of claim 1; and
one or more databases that store treatment data and patient data.
